(19) United States
(12) Patent Application Publication
(10) Pub. No.: US 2009/0318470 A1
(43) Pub. Date: Dec. 24, 2009

(54) 1-SUBSTITUTED-3-(NAPHTHALEN-1-YLSULFONYL)-5-(PIPERAZIN-1-YL)-1H-INDAZOLE COMPOUNDS AS 5-HYDROXYTRYPTAMINE-6 LIGANDS

(75) Inventors: Kevin Guangcheng Liu, West Windsor, NJ (US); Albert Jean Robichaud, Ringoes, NJ (US); Jennifer Rebecca Lo, South Brunswick, NJ (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/487,690

(22) Filed: Jun. 19, 2009

Related U.S. Application Data
(60) Provisional application No. 61/074,166, filed on Jun. 20, 2008.

(51) Int. Cl. A61K 31/496; C07D 403/10; A61P 25/00

(52) U.S. Cl. 514/254.06; 544/371

(57) ABSTRACT

The disclosure is directed to compounds of Formula I:

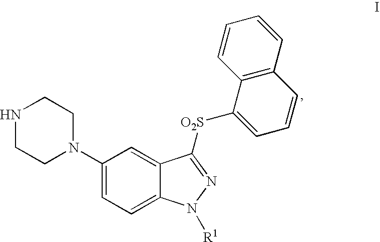

processes for their preparation, treatment of central nervous system (CNS) disorders and methods for the modulation of 5HT₆ activity.

### FIELD OF THE INVENTION

The present invention is directed to 1-substituted-3-(napthalen-1-ylsufonyl)-5-(piperazin-1-yl)-1H-indazole compounds, processes for their preparation, their use for modulation of 5HT₆ activity and methods for treatment of central nervous system (CNS) disorders.

### BACKGROUND OF THE INVENTION

Serotonin (5-hydroxytryptamine) (5-HT) receptors play a critical role in many physiological and behavioral functions in humans and animals. These functions are mediated through various 5-HT receptors distributed throughout the body. There are now approximately fifteen different human 5-HT receptor subtypes that have been cloned, many with well-defined roles in humans. A recently identified 5-HT receptor subtype is the 5-HT₆ receptor, first cloned from rat tissue in 1993 (Monsma, F. J.; Shen, Y.; Ward, R. P.; Hamblin, M. W. Molecular Pharmacology 1993, 43, 320-327) and subsequently from human tissue (Kohen, R.; Metcalf, M. A.; Khan, N.; Druck, T.; Huebner, K.; Sibley, D. R. Journal of Neurochemistry 1996, 66, 47-56). The receptor is a G-protein coupled receptor (GPCR) positively coupled to adenylate cyclase (Ruat, M.; Traiffort, E.; Arrang, J-M.; Tardivel-Lacombe, L.; Diaz, L.; Leurs, R.; Schwartz, J-C. Biochemical Biophysical Research Communications 1993, 193, 268-276). The receptor is found almost exclusively in the central nervous system (CNS) areas both in rat and in human. In situ hybridization studies of the 5-HT₆ receptor in rat brain using mRNA indicate principal localization in the areas of 5-HT projection including striatum, nucleus accumbens, olfactory tubercle, and hippocampal formation (Ward, R. P.; Hamblin, M. W.; Lachowicz, J. E.; Hoffman, B. J.; Sibley, D. R.; Dorsa, D. M. Neuroscience 1995, 64, 1105-1111).

There are many potential therapeutic uses for 5-HT₆ ligands in humans based on direct effects and on indications from available scientific studies. These studies include the localization of the receptor, the affinity of ligands with known in vivo activity, and various animal studies conducted so far.

One potential therapeutic use of modulators of 5-HT₆ receptor function is in the enhancement of cognition and memory in human diseases such as Alzheimer's. The high levels of receptor found in important structures in the forebrain, including the caudate/putamen, hippocampus, nucleus accumbens, and cortex suggest a role for the receptor in memory and cognition since these areas are known to play a vital role in memory (Gerard, C.; Martres, M.-P.; Lefevre, K.; Miquel, M. C.; Verge, D.; Lanfumey, R.; Doucet, E.; Hamon, M.; El Mestikawy, S. Brain Research, 1997, 746, 207-219). The ability of known 5-HT₆ receptor ligands to enhance cholinergic transmission also supported the potential cognition use (Bentley, J. C.; Boursson, A.; Boess, F. G.; Kone, F. C.; Marsden, C. A.; Petit, N.; Sleight, A. J. British Journal of Pharmacology, 1999, 126(7), 1537-1542). Studies have found that a known 5-HT₆ selective antagonist significantly increased glutamate and aspartate levels in the frontal cortex without elevating levels of noradrenaline, dopamine, or 5-HT. This selective elevation of neurochemicals known to be involved in memory and cognition point to a role for 5-HT₆ ligands in cognition (Dawson, L. A.; Nguyen, H. Q.; Li, P. British Journal of Pharmacology, 2000, 130(1), 23-26). Animal studies of memory and learning with a known selective 5-HT₆ antagonist found positive effects (Rogers, D. C.; Hatcher, P. D.; Hagan, J. J. Society of Neuroscience, Abstracts 2000, 26, 680).

A related potential therapeutic use for 5-HT₆ ligands, particularly antagonists, is the treatment of attention deficit disorders (ADD) and Attention Deficit Hyperactivity Disorder (ADHD) in both children and adults. Ernst, M; Zametkin, A. J.; Matochik, J. H.; Jons, P. A.; Cohen, R. M. Journal of Neuroscience 1998, 18(15), 5901-5907).

5-HT₆ ligands also show potential for the treatment of schizophrenia and depression. For example, clozapine (an effective clinical antipsychotic) has high affinity for the 5-HT₆ receptor subtype. Also, several clinical antidepressants have high affinity for the receptor as well and act as antagonists at this site (Branchek, T. A.; Blackburn, T. P. Annual Reviews in Pharmacology and Toxicology 2000, 40, 319-334).

Further, recent in vivo studies in rats indicate 5-HT₆ modulators may be useful in the treatment of movement disorders including epilepsy (Stean, T.; Routledge, C.; Upton, N. British Journal of Pharmacology 1999, 127 Proc. Supplement 131P and Routledge, C.; Bromidge, S. M.; Moss, S. F.; Price, G. W.; Hirst, W.; Newman, H.; Riley, G.; Gager, T.; Stean, T.; Upton, N.; Clarke, S. E.; Brown, A. M. British Journal of Pharmacology 2000, 130(7), 1606-1612).

Taken together, the above studies strongly suggest that compounds which are 5-HT₆ receptor modulators, i.e. ligands, may be useful for therapeutic indications including: the treatment of symptoms associated with Alzheimer's disease, such as dementia, a deficit in memory, cognition, and learning; the treatment of personality disorders such as schizophrenia; the treatment of behavioral disorders, e.g., anxiety, depression and obsessive compulsive disorders; the treatment of ADD and ADHD; the treatment of motion or motor disorders such as Parkinson's disease and epilepsy; the treatment of diseases associated with neurodegeneration such as stroke and head trauma; or withdrawal from drug addiction including addiction to nicotine, alcohol, among others.

Because 5HT₆ receptors are located almost exclusively in the brain, modulation of the receptors by parenterally administered drugs requires that the drugs cross the blood brain barrier. The blood brain barrier is composed of brain capillary endothelial cells with continuous tight junctions making it virtually impossible for compounds to enter into the brain around the cells. J. Bryan, Pharmaceutical Journal, v 273 (2004) 475-476. Instead, access to the brain is limited to passive diffusion or active transport through the endothelial cells. G&G, Pharmaceutical Basis of Therapeutics, 10^{th} Ed at page 10. Accordingly, bioavailable parenterally administered compounds affecting 5HT₆ activity must not only possess favorable solubility profiles in order to successfully enter the blood stream, but they also need to cross the blood brain barrier in order to target the 5HT₆ receptors.

In addition to bioavailability, toxicity associated with metabolism and compound degradation is a major concern for any potential drug candidate, especially for drugs capable of affecting the central nervous system.

Advantageously, this invention provides compounds that are capable of modulating 5-HT₆ receptor activity, have bioavailability, and have improved microsomal stability.

The invention provides compounds useful as therapeutic agents in the treatment of a variety of conditions related to or affected by 5HT₆ receptor activity, including psychoses (e.g., schizophrenia, anxiety, or depression), motor disorders (e.g., Parkinson's disease), anxiety, depression, drug addiction, obsessive compulsive disorder, attention deficit disorder, or any condition which is known to be related to or affected by the 5-HT₆ receptor.

These and other features of this invention will become more apparent by the detailed description set forth hereinbelow.

### SUMMARY OF THE INVENTION

The present invention provides 5HT₆ antagonists that are bioavailable, capable of crossing the blood brain barrier and have increased microsomal stability.

In one embodiment, the invention provides a compound of Formula I:

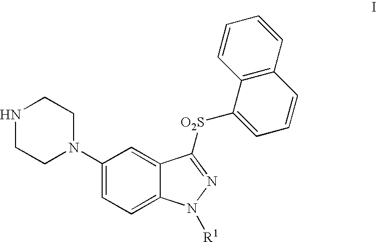

wherein,

R¹ is selected from the group consisting of C₆-C₁₀ aryl, C₁-C₆ alkyl, C₇-C₁₄ arylalkyl and C₁-C₆ acyl, each substituted with 0-4 substituents independently selected from the group consisting of C₁-C₄ alkyl, C₃-C₆ cycloalkyl, C₂-C₆ alkenyl, C₂-C₆ alkynyl, halo, nitro, cyano, hydroxy, phenyl, 5-7 membered heterocyclyl, 5-7 membered heteroaryl, —N(R^{a})₂, —C(O)R^{b}, —OR^{c} and —S(O)ₚR^{d};

each R^{a} is independently H, C₁-C₄ alkyl optionally substituted with halo, —CHO, —C(O)(C₁-C₄ alkyl), or —CO₂(C₁-C₄ alkyl);

each R^{b} is independently H, —OH, —O(C₁-C₄), C₁-C₄ alkyl optionally substituted with halo, —NH₂, —NH(C₁-C₄ alkyl), or —N(C₁-C₄ alkyl)₂;

each R^{c} is independently H, C₁-C₄ alkyl optionally substituted with halo, —CHO or —C(O)(C₁-C₄ alkyl);

each R^{d} is independently C₁-C₄ alkyl optionally substituted with halo or —OH; and

each p is independently 0, 1 or 2; or

a tautomer or pharmaceutically acceptable salt thereof.

In another embodiment, the invention provides methods of modulating 5HT₆ activity comprising contacting the compound of Formula I with a 5HT₆ receptor. In another aspect, the invention provides treating a subject suffering from a CNS disorder related to 5HT₆ activity or affected by 5HT₆ modulation, more particularly inhibition, comprising administering the compound of Formula I to the subject in need thereof.

Another embodiment of the invention provides a process for the preparation of the compound of Formula I.

Another embodiment of the present invention provides use of a compound of any one of the embodiments described herein for the treatment of a disease or disorder described herein. More particularly, the present invention provides for use of a compound of any one of the embodiments described herein in the manufacture of a medicament for the treatment of a disease or disorder described herein.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### DETAILED DESCRIPTION OF THE INVENTION

Terms used throughout the application and in the claims are defined below.

“Acyl” refers to the groups H—C(O)—, alkyl-C(O)—, alkenyl-C(O)—, alkynyl-C(O)—, cycloalkyl-C(O)—, cycloalkenyl-C(O)—, aryl-C(O)—, 5-7 membered heteroaryl-C(O)—, 5-7 membered heterocyclic-C(O)—, wherein alkyl, alkenyl, alkynyl, cycloalkyl, cycloalkenyl, aryl, heteroaryl, and heterocyclic are as defined herein. Acyl includes the “acetyl” group CH₃C(O)—.

“Alkyl” refers to monovalent saturated aliphatic hydrocarbyl groups having from 1 to 6 carbon atoms (C₁-C₆ alkyl), and preferably 1 to 4 carbon atoms (C₁-C₄ alkyl). This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), and t-butyl ((CH₃)₃C—).

“Amino” refers to the group —NH₂.

“Aryl” or “Ar” refers to a monovalent aromatic carbocyclic group of from 6 to 10 carbon atoms having a single ring (e.g., phenyl) or multiple condensed rings (e.g., naphthyl). The condensed rings may or may not all be aromatic (e.g., 1,2,3,4-tetrahydronaphthyl) provided that the point of attachment is at an aromatic carbon atom. Preferred aryl groups are phenyl and naphthyl.

“Arylalkyl” refers to an aryl group as defined herein appended at any suitable position to an alkyl group, wherein the point of attachment to the base-compound is at the alkyl group. Preferred arylalkyl groups have 7 to 14 carbon atoms (C₇-C₁₄ arylalkyl), more preferably the aryl portion is phenyl (C₆) and the alkyl portion is C₁-C₂. In such embodiments the group is C₇-C₉ arylalkyl. Examples of arylalkyl groups include benzyl and phenethyl.

“Alkenyl” refers to alkenyl groups having from 2 to 6 carbon atoms (C₂-C₆ alkenyl) and preferably 2 to 4 carbon atoms (C₂-C₄ alkenyl) and having at least 1 and preferably from 1 to 2 sites of alkenyl unsaturation. Such groups are exemplified, for example, by vinyl, allyl, and but-3-en-1-yl.

“Alkynyl” refers to alkynyl groups having from 2 to 6 carbon atoms (C₂-C₆ alkynyl) and preferably 2 to 3 carbon atoms (C₂-C₃ alkynyl) and having at least 1 and preferably from 1 to 2 sites of alkynyl unsaturation. Such groups are exemplified, for example, by propargyl.

“Cyano” refers to the group —CN.

“Cycloalkyl” refers to cyclic alkyl groups of from 3 to 10 carbon atoms having single or multiple cyclic rings including fused, bridged, and spiro ring systems. Preferred cycloalkyl groups have 3 to 6 carbon atoms (C₃-C₆ cycloalkyl). Examples of suitable cycloalkyl groups include, for instance, adamantyl, cyclopropyl, cyclobutyl, cyclopentyl, cyclohexyl, and cyclooctyl.

“Halo” or “halogen” refers to fluoro, chloro, bromo and iodo.

“Haloalkyl” refers to an alkyl group as defined herein wherein at least one hydrogen atom on the alkyl group is replaced with a halo group (e.g. chloroethyl or trifluoromethyl).

“Hydroxy” or “hydroxyl” refers to the group —OH.

5 to 7 membered “heteroaryl” refers to an aromatic group of from 5 to 7 atoms, with 1 to 6 carbon atoms and 1 to 4 heteroatoms selected from the group consisting of oxygen, nitrogen and sulfur within the ring. In one embodiment, the nitrogen and/or the sulfur ring atom(s) of the heteroaryl group are optionally oxidized to provide for the N-oxide (N→O), sulfinyl, or sulfonyl moieties. Preferred heteroaryls include pyridinyl, pyrrolyl, thiophenyl, and furanyl.

5 to 7 membered “heterocyclic” or “heterocyclyl” refers to a saturated or unsaturated group having a single non-aromatic ring with from 1 to 6 carbon atoms and from 1 to 4 hetero atoms selected from the group consisting of nitrogen, sulfur or oxygen within the ring. In one embodiment, the nitrogen and/or sulfur atom(s) of the heterocyclic group are optionally oxidized to provide for the N-oxide, sulfinyl, or sulfonyl moieties. Examples of heterocyclic groups include, but are not limited to, piperazinyl, morpholinyl, thiomorpholinyl (also referred to as thiamorpholinyl), 1,1-dioxothiomorpholinyl, piperidinyl, pyrrolidinyl, and tetrahydrofuranyl.

“Nitro” refers to the group —NO₂.

“Oxo” refers to the atom (═O) or (—O⁻). As an activating group, ‘oxo’ groups are amenable to reductive amination by nucleophilic amine groups to form alkylamino or aminoalkyl substituents. Preferably, the reductive amination step takes place in the presence of a boron-containing reducing agent.

“Spirocyclyl” refers to divalent saturated cyclic group from 3 to 10 carbon atoms having a cycloalkyl or heterocyclyl ring with a spiro union (the union formed by a single atom which is the only common member of the rings) as exemplified by the following structure:

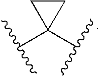

“Stereoisomer” or “stereoisomers” refer to compounds that differ in the chirality or atomic connectivity at one or more stereocenters. Stereoisomers include enantiomers, diastereomers as well as cis-trans (E/Z) isomerism.

“Tautomer” refers to alternate forms of a compound that differ in the position of a proton, such as enol-keto and imine-enamine tautomers, or the tautomeric forms of heteroaryl groups containing a ring atom attached to both a ring —NH— moiety and a ring ═N— moiety such as pyrazoles, imidazoles, benzimidazoles, triazoles, and tetrazoles.

“Patient” or “subject” refers to mammals and includes humans and non-human mammals, such as dogs, cats, mice, rats, cows, rabbits and monkeys. Preferred patient or subject is human.

“Pharmaceutically acceptable salt” refers to pharmaceutically acceptable salts of a compound, which salts are derived from a variety of organic and inorganic counter ions well known in the art and include, by way of example only, acetate, aluminum, amsonate (4,4-diaminostilbene-2,2-disulfonate), benzathine (N,N′-dibenzylethylenediamine), benzenesulfonate, benzoate, bicarbonate, bismuth, bisulfate, bitartrate, borate, bromide, butyrate, calcium, calcium edetate, camsylate(camphorsulfonate), carbonate, chloride, choline, citrate, clavulariate, diethanolamine, dihydrochloride, diphosphate, edetate, edisylate(camphorsulfonate), esylate(ethanesulfonate), ethylenediamine, fumarate, gluceptate(glucoheptonate), gluconate, glucuronate, glutamate, hexafluorophosphate, hexylresorcinate, hydrabamine (N,N′-bis(dehydroabietyl)ethylenediamine), hydrobromide, hydrochloride, hydroxynaphthoate, 1-hydroxy-2-naphthoate, 3-hydroxy-2-naphthoate, iodide, isothionate(2-hydroxyethanesulfonate), lactate, lactobionate, laurate, lauryl sulfate, lithium, magnesium, malate, maleate, mandelate, meglumine(1-deoxy-1-(methylamino)-D-glucitol), mesylate, methyl bromide, methylnitrate, methylsulfate, mucate, napsylate, nitrate, N-methylglucamine ammonium salt, oleate, oxalate, palmitate, pamoate(4,4′-methylenebis-3-hydroxy-2-naphthoate, or embonate), pantothenate, phosphate, picrate, polygalacturonate, potassium, propionate, p-toluenesulfonate, salicylate, sodium, stearate, subacetate, succinate, sulfate, sulfosaliculate, suramate, tannate, tartrate, teoclate(8-chloro-3,7-dihydro-1,3-dimethyl-1H-purine-2,6-dione), triethiodide, tromethamine(2-amino-2-(hydroxymethyl)-1,3-propanediol), valerate, and zinc salts. Certain compounds of the invention can form pharmaceutically acceptable salts with various amino acids as well. For a review on pharmaceutically acceptable salts see Berge et al., 66 J. Pharm. Sci. 1-19 (1977), incorporated herein by reference.

The pharmaceutically acceptable salts are prepared by cotacting a compound, such as the compound of formula (I) with an acid or salt such as, hydrochloric acid, hydrobromic acid, acetic acid, phosphoric acid, boric acid, perchloric acid, tartaric acid, maleic acid, citric acid, methanesulfonic acid, ascorbic acid, sodium iodide and the like. A solvent employed may be selected from ketones such as acetone, diethyl ketone, methyl ethyl ketone or their mixtures, methanol, ethanol, n-hexane, ethylacetate, benzene, diethylamine, formaldehyde, chloroform, dichloromethane or mixture thereof.

“Treating” or “treatment” of a disease in a subject in need thereof refers to: inhibiting the disease or arresting its development; ameliorating a symptom of the disease; or causing regression of the disease. Accordingly, “treatment of Alzheimer's disease” as used herein encompasses amelioration of symptoms associated with Alzheimer's disease, such as the amelioration of Alzheimer's-related dementia.

“Modulating 5HT₆ receptor activity” refers to affecting (i.e. inhibition or stimulation) processes or signaling events associated with the 5HT₆ receptor. Specifically, inhibition of 5HT₆ increases levels of acetylcholine and glutamate in the brain, whereas 5HT₆ receptor agonism or stimulation results in increased cellular cAMP.

A “CNS disease” or “CNS disorder” is a disease or disorder affecting or originating in the central nervous system, preferably a disease related to 5HT₆ activity or affected by 5HT₆ modulation. Particular CNS diseases or disorder include psychoses, anxiety, depression, epilepsy, migraine, cognitive disorders, sleep disorders, feeding disorders, anorexia, bulimia, binge eating disorders, panic attacks, disorders resulting from withdrawal from drug abuse, schizophrenia, gastrointestinal disorders, irritable bowel syndrome, memory disorders, obsessive compulsive disorders, Alzheimer's disease, ADD, ADHD, Restless Legs Syndrome (RLS), Parkinson's disease, Huntington's chorea, schizophrenia, attention deficit hyperactive disorder, neurodegenerative diseases characterized by impaired neuronal growth, and pain. A “CNS disease” or “CNS disorder” as used herein also includes the symptom of another disease, such as dementia and other cognitive disorders associated with Alzheimer's disease.

Unless indicated otherwise, the nomenclature of substituents that are not explicitly defined herein are arrived at by naming from left to right the terminal portion of the functionality followed by the adjacent functionality toward the point of attachment. For example, the substituent “arylalkyloxycabonyl” refers to the group (aryl)-(alkyl)—O—C(O)—.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group etc.) are not intended for inclusion herein. In such cases, the maximum number of such substitutions is three. For example, serial substitutions of substituted aryl groups with two other substituted aryl groups are limited to -substituted aryl-(substituted aryl)-substituted aryl.

Similarly, it is understood that the above definitions are not intended to include impermissible substitution patterns (e.g., methyl substituted with 5 fluoro groups). Such impermissible substitution patterns are well known to the skilled artisan.

At various places in the present specification, substituents of compounds are disclosed in groups or in ranges. It is specifically intended that the description include each and every individual subcombination of the members of such groups and ranges. For example, the term “C₁₋₆ alkyl” is specifically intended to individually disclose C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ and alkyl. By way of another example, the term “5-7 membered heteroaryl or heterocyclyl group” is specifically intended to individually disclose a heteroaryl or heterocyclyl group having 5, 6, 7, 5-7, and 5-6 ring atoms.

One aspect of the invention provides a compound of Formula I:

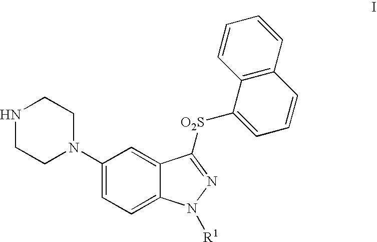

wherein,

R¹ is selected from the group consisting of C₆-C₁₀ aryl, C₁-C₆ alkyl, C₇-C₁₄ arylalkyl and C₁-C₆ acyl, each substituted with 0-4 substituents independently selected from the group consisting of C₁-C₄ alkyl, C₃-C₆ cycloalkyl, C₂-C₆ alkenyl, C₂-C₆ alkynyl, halo, nitro, cyano, hydroxy, phenyl, 5-7 membered heterocyclyl, 5-7 membered heteroaryl, —N(R^{a})₂, —C(O)R^{b}, —OR^{c} and —S(O)ₚR^{d};

each R^{a} is independently H, C₁-C₄ alkyl optionally substituted with halo, —CHO, —C(O)(C₁-C₄ alkyl), or —CO₂(C₁-C₄ alkyl);

each R^{b} is independently H, —OH, —O(C₁-C₄), C₁-C₄ alkyl optionally substituted with halo, —NH₂, —NH(C₁-C₄ alkyl), or —N(C₁-C₄ alkyl)₂;

each R^{c} is independently H, C₁-C₄ alkyl optionally substituted with halo, —CHO or —C(O)(C₁-C₄ alkyl);

each R^{d} is independently C₁-C₄ alkyl optionally substituted with halo or —OH; and

each p is independently 0, 1 or 2; or

a tautomer or pharmaceutically acceptable salt thereof.

In another embodiment, R¹ is C₁-C₆ alkyl, C₁-C₆ acyl, or C₇-C₉ arylalkyl, each optionally substituted with 1, 2, or 3 halo; or a tautomer or pharmaceutically acceptable salt thereof.

In another embodiment, R¹ is C₁-C₆ alkyl or C₇-C₉ arylalkyl, each optionally substituted with 1, 2, or 3 halo; or a tautomer or pharmaceutically acceptable salt thereof.

In another embodiment, R¹ is C₁-C₆ alkyl; or tautomer or pharmaceutically acceptable salt thereof. More particularly, R¹ is methyl, ethyl, n-propyl, isopropyl, n-butyl, isobutyl, or tert-butyl; or a tautomer or pharmaceutically acceptable salt thereof. More particular still, R¹ is methyl, ethyl, n-propyl, or isopropyl; or a tautomer or pharmaceutically acceptable salt thereof.

In another embodiment, R¹ is C₇-C₉ arylalkyl optionally substituted with 1, 2, or 3 halo;

a tautomer or pharmaceutically acceptable salt thereof. More particularly, R¹ is benzyl optionally substituted with 1, 2, or 3 halo; or a tautomer or pharmaceutically acceptable salt thereof.

A particular aspect of the invention provides a compound selected from the group consisting of:

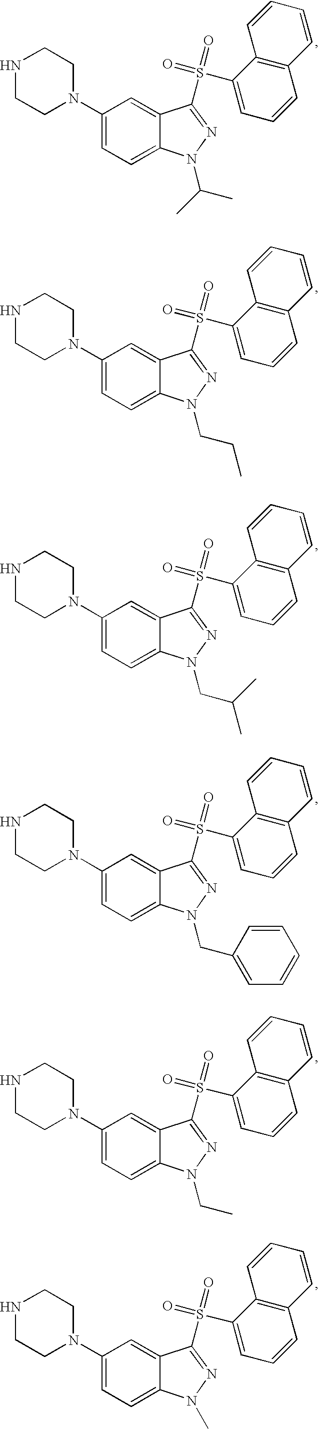

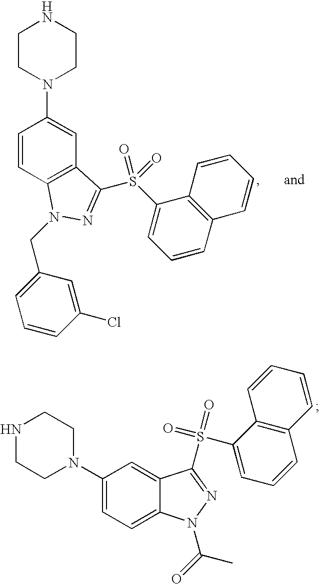

or a tautomer or pharmaceutically acceptable salt thereof.

A particular embodiment provides a compound having the structure:

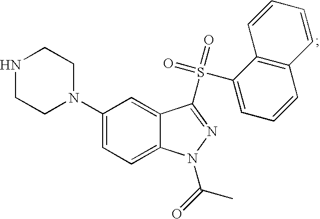

or a tautomer or pharmaceutically acceptable salt thereof.

A particular embodiment provides a compound having the structure:

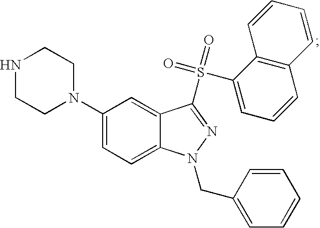

or a tautomer or pharmaceutically acceptable salt thereof.

A particular embodiment provides a compound having the structure:

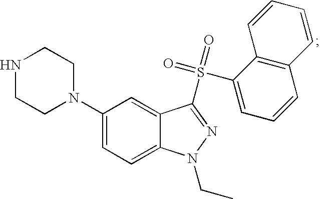

or a tautomer or pharmaceutically acceptable salt thereof.

A particular embodiment provides a compound having the structure:

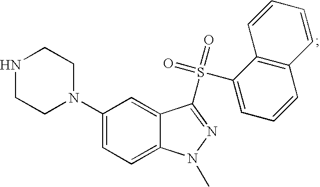

or a tautomer or pharmaceutically acceptable salt thereof.

A particular embodiment provides a compound having the structure:

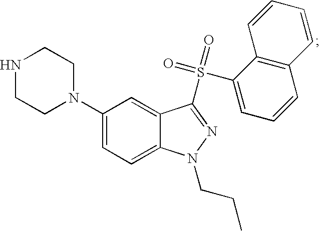

or a tautomer or pharmaceutically acceptable salt thereof.

A particular embodiment provides a compound having the structure:

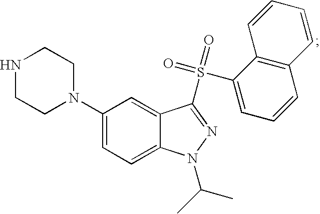

or a tautomer or pharmaceutically acceptable salt thereof.

In another embodiment, the compound of any of the embodiments of the present invention exists as a pharmaceutically acceptable salt. More particularly, the pharmaceutically acceptable salt is hydrochloride (HCl). More particular still, the pharmaceutically acceptable salt of any of the compounds is mono or bis hydrochloride (HCl).

Another embodiment of the invention provides pharmaceutical compositions comprising compounds or pharmaceutically acceptable salts of the compounds of the present Formula I and a pharmaceutically acceptable carrier.

Another embodiment of the invention provides a method of modulating 5HT₆ receptor activity in a subject, comprising administering to the subject in need thereof a compound of Formula I, wherein 5HT₆ receptor activity is modulated in the subject.

More particularly, the compound is administered parenterally to the subject.

More particularly, the compound is capable of crossing the blood brain barrier in the subject.

In another embodiment, the compound is administered in an amount sufficient to provide about 1 to about 4000 ng/mL of the compound to brain: tissue, plasma, blood, central spinal fluid (CSF), or intraspinal fluid (ISF) of the subject within 24 hours after administration.

Another embodiment of the invention provides a method for modulating 5HT₆ activity in a subject, comprising administering a unit dose of a compound of Formula I, wherein each unit dose of the medicament is sufficient to provide at least one of (a) a Cmax of about 20 to 4000 ng/mL of the compound in a subject's plasma or a Cmax of about 40 to 8000 ng/mL of the compound in the subject's blood when it is administered to the subject, (b) about 10 to 2,000 ng/mL of the compound in a subject's plasma 24 hours after administration or about 20 to 4,000 ng/mL of the compound in the subject's blood 24 hours after administration to the subject, or (c) an AUC of about 500 to 60,000 ng*h/mL of the compound in a subject's plasma or about 750 to 120,000 ng*h/mL of the compound in the subject's blood when it is administered to the subject.

More particularly, the compound is administered parenterally to the subject. More particular still, the compound is administered orally to the subject. More particularly, the blood or plasma is located in the brain.

In a more particular embodiment, each unit dose is sufficient to provide at least one of (a) a Cmax of about 50 to 500 ng/mL of the compound in the subject's plasma or a Cmax of about 100 to 1000 ng/mL of the compound in the subject's blood, about 20 to 1,000 ng/mL of the compound in the subject's plasma, 24 hours after administration or about 40 to 2,000ng/mL of the compound in the subject's blood 24 hours after administration, or (c) an AUC of about 1,000 to 30,000 ng*h/mL of the compound in the subject's plasma or about 1,500 to 60,000ng*h/mL of the compound in the subject's blood.

In another more particular embodiment, each unit dose comprises from 0.25 to 30 mg/kg of the compound, tautomer, and/or salts based on the body weight of the subject.

In another embodiment of the invention, the compound exhibits an oral bioavailability of greater than 5%. More particularly, the compound exhibits an oral bioavailability of greater than 10%, 15%, 20%, 25%, 30%, 35%, 40%, 45%, 50%, 55%, or 60%.

In another embodiment, the compound exhibits a brain plasma ratio of greater than 0.05 0.1, 0.2, 0.3, 0.4, 0.5, 0.6, 0.7 or 0.8 ng/mL.

In another embodiment, the compounds exhibit an AUC_{0-inf} of greater than 400, 600 or 800 hr*ng/mL in Male Long Evans Rats following a single IV bolus. In another embodiment, the compounds exhibit an AUC_{0-inf} of greater than 200, 400, 1000 or 2000 hr*ng/mL in Male Long Evans Rats following a single oral dose of 10 mg/kg. In another embodiment, the compounds exhibit AUC_{last-CNS} penetration of greater than 100 or 300 ng/mL in brain and/or greater than 200, 300 or 400 in plasma in Male Long Evans Rats following a single intraperitoneal dose of 10 mg/mL.

In another embodiment, the compound exhibits microsomal stability in rats or humans of greater than 5 minutes, 10 minutes, 20 minutes or 30 minutes.

Another embodiment of the invention provides a method of treating a central nervous system (CNS) disease or disorder comprising administering to the subject in need thereof a compound described herein, particularly a compound of Formula I.

In another embodiment, the subject is suffering from psychoses, anxiety, depression, epilepsy obsessive compulsive disorders, migraine, cognitive disorders, sleep disorders, feeding disorders, anorexia, bulimia, binge eating disorders, panic attacks, disorders resulting from withdrawal from drug abuse, schizophrenia, gastrointestinal disorders, irritable bowel syndrome, memory disorders, Alzheimer's disease, Parkinson's disease, Huntington's chorea, schizophrenia, attention deficit hyperactive disorder, neurodegenerative diseases characterized by impaired neuronal growth, or pain.

The present invention further provides methods and processes for the preparation of the compounds described herein. In one embodiment, the present invention provides a process for the preparation of compounds of Formula I as depicted in Scheme 1.

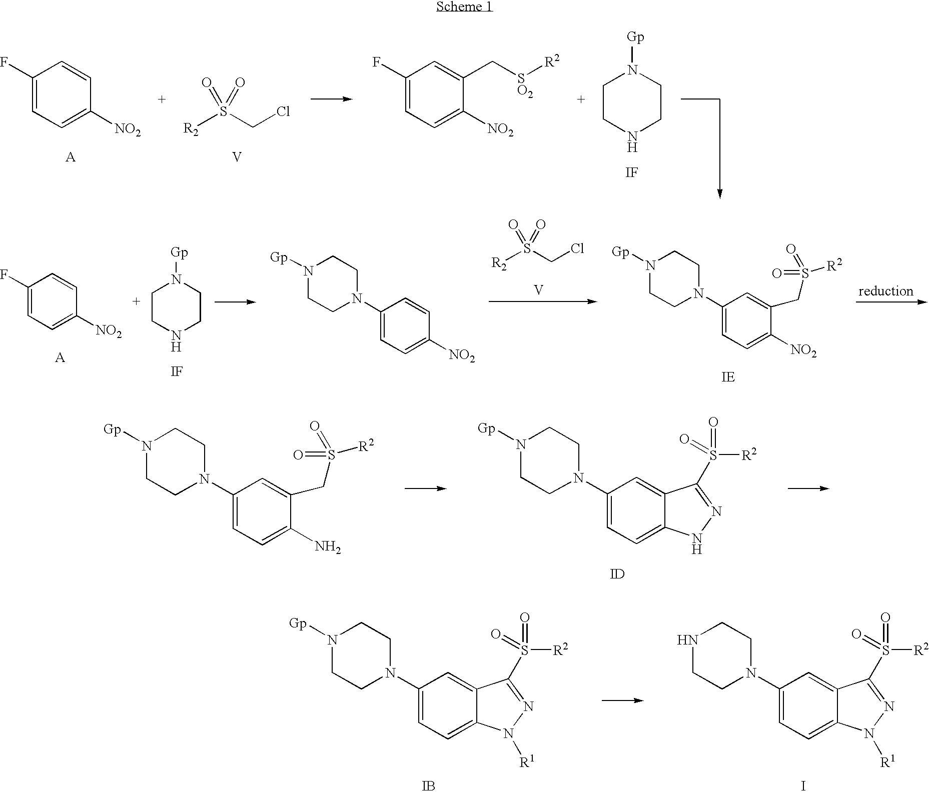

In Scheme 1, R² is naphthyl and compound IE is prepared either from initially reacting the compound V with compound A followed by S_{N}Ar with IF; or initial S_{N}Ar of A with IF followed by a reaction with V. A number of protecting groups (G_{P}) such as Cbz and Boc can be employed for the synthesis. Removal of the protecting groups from IB using standard procedures (see Greene et. al. Protective Groups in Organic Synthesis) affords compounds of Formula I.

In the processes of preparation described herein, it is contemplated that variables, such as protecting groups, can be modified or substituted in between recited steps, so long as the modified group falls within the claimed genus. For example, in ID, a protecting group (G_{P}) on a compound may be Cbz, whereas in later steps, such as IB, the G_{P} group at the same position may be Boc. In such instances, a 2-step, deprotecting-protecting reaction will occur.

The term “protecting group” or “G_{P}” with respect to amine groups, hydroxyl groups and sulfhydryl groups refers to forms of these functionalities which are protected from undesirable reaction with a protecting group known to those skilled in the art, such as those set forth in Protective Groups in Organic Synthesis, Greene, T. W.; Wuts, P. G. M., John Wiley & Sons, New York, N.Y., (3rd Edition, 1999), the entire disclosure of which is herein incorporated by reference, which protecting groups can be added or removed using the procedures set forth therein. Examples of protected hydroxyl groups include, but are not limited to, silyl ethers such as those obtained by reaction of a hydroxyl group with a reagent such as, but not limited to, t-butyldimethyl-chlorosilane, trimethylchlorosilane, triisopropylchlorosilane, triethylchlorosilane; substituted methyl and ethyl ethers such as, but not limited to methoxymethyl ether, methythiomethyl ether, benzyloxymethyl ether, t-butoxymethyl ether, 2-methoxyethoxymethyl ether, tetrahydropyranyl ethers, 1-ethoxyethyl ether, allyl ether, benzyl ether; esters such as, but not limited to, benzoylformate, formate, acetate, trichloroacetate, and trifluoracetate. Examples of protected amine groups include, but are not limited to, amides such as, formamide, acetamide, trifluoroacetamide, and benzamide; carbamates; e.g. Boc; imides, such as phthalimide, Fmoc, Cbz, PMB, benzyl, and dithiosuccinimide; and others. Examples of protected or capped sulfhydryl groups include, but are not limited to, thioethers such as S-benzyl thioether, and S-4-picolyl thioether; substituted S-methyl derivatives such as hemithio, dithio and aminothio acetals; and others.

As used herein, an “an activating group” is a group that, when bound to a center, increases the reactivity at that center. Non-limiting examples of an activating group include a substituent bound to an electrophilic center and capable of being displaced by a nucleophile; a substituent bound to a nucleophilic center and capable of being displaced by an electrophile; a substituent capable of being displaced by a radical; or a substituent bound to a center wherein, following gain or loss of an electron, the substituent is capable of leaving as an anion or cation with formation of a radical at the center.

As used herein, “activating” a compound refers to reacting the compound at a center with a reagent to introduce at the center an activating group, wherein the activating group is optionally converted to another activating group in one or more steps. Examples of activating include halogenation at a carbon center, optionally followed by hydroboration wherein the halogen group is converted to an optionally substituted borane; tosylation, mesylation, or triflation at an oxygen center; and nitration at a carbon center optionally followed by reduction of the nitro group to an amino group and conversion of the amino group to a diazo group.

The term “deprotecting” refers to removal of a protecting group, such as removal of a benzyl or BOC group bound to an amine. Deprotecting may be preformed by heating and/or addition of reagents capable of removing protecting groups. In preferred embodiments, the deprotecting step involves addition of an acid, base, reducing agent, oxidizing agent, heat, or any combination thereof. One preferred method of removing BOC groups from amino groups is to add HCl or TFA to a solution. Many deprotecting reactions are well known in the art and are described in Protective Groups in Organic Synthesis, Greene, T. W., John Wiley & Sons, New York, N.Y., (1st Edition, 1981), the entire disclosure of which is herein incorporated by reference.

As used herein, a diazotizing reagent is a reagent capable of converting an amine to a diazo compound. Diazotizing reagents include nitrous acid (generated in situ from sodium nitrite and a strong acid, such as hydrochloric acid, sulfuric acid, or HBF₄), nitrosyl sulfuric acid (sodium nitrite dissolved in an excess of sulfuric acid) in sulfuric acid or sulfuric acid in a lower carboxylic acid such as acetic acid or proprionic acid, and isopentyl nitrite in trifluoroacetic acid.

Another aspect of the invention provides a process for the preparation of a compound of Formula I:

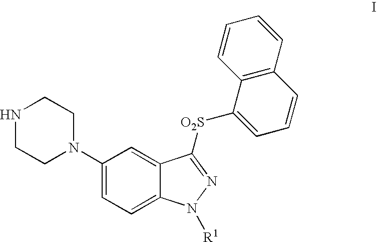

wherein,

R¹ is selected from the group consisting of C₆-C₁₀ aryl, C₁-C₆ alkyl, C₇-C₁₄ arylalkyl and C₁-C₆ acyl, each substituted with 0-4 substituents independently selected from the group consisting of C₁-C₄ alkyl, C₃-C₆ cycloalkyl, C₂-C₆ alkenyl, C₂-C₆ alkynyl, halo, nitro, cyano, hydroxy, phenyl, 5-7 membered heterocyclyl, 5-7 membered heteroaryl, —N(R^{a})₂, —C(O)R^{b}, —OR^{c} and —S(O)ₚR^{d};

each R^{a} is independently H, C₁-C₄ alkyl optionally substituted with halo, —CHO, —C(O)(C₁-C₄ alkyl), or —CO₂(C₁-C₄ alkyl);

each R^{b} is independently H, —OH, —O(C₁-C₄), C₁-C₄ alkyl optionally substituted with halo, —NH₂, —NH(C₁-C₄ alkyl), or —N(C₁-C₄ alkyl)₂;

each R^{c} is independently H, C₁-C₄ alkyl optionally substituted with halo, —CHO or —C(O)(C₁-C₄ alkyl);

each R^{d} is independently C₁-C₄ alkyl optionally substituted with halo or —OH; and

each p is independently 0, 1 or 2; or

a tautomer or pharmaceutically acceptable salt thereof

the process comprising:

reacting R¹-G_{A} or R¹═O with a compound of Formula IA:

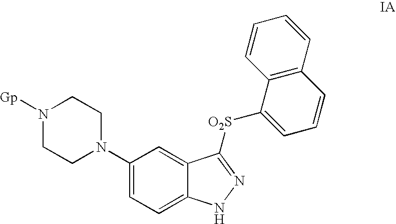

wherein,

G_{A} is an activating group; and

G_{P} is a protecting group;

to form a compound of Formula IB:

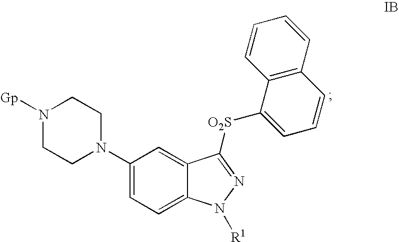

and

deprotecting the compound of Formula IB;

wherein the compound of Formula I is formed.

In another embodiment, G_{A} is halo, tosylate, mesylate or triflate. More particularly, G_{A} is iodo.

In another embodiment, G_{P} is t-butoxycarbonyl (Boc), benzyl, acetyl, p-methoxybenzyl (PMB), C₁-C₆ alkyl, 9-fluoroenylmethoxycarbonyl (Fmoc), benzyloxycarbonyl (Cbz), trifluoroacetyl, tosyl or trityl.

In another embodiment, reacting step is performed in the presence of a base. More particularly, the base is potassium tertiary butoxide (KOₜBu).

In another embodiment, the deprotecting step is performed in the presence of an acid. More particularly, the acid is trifluoroacetic acid (TFA) or hydrochloric acid (HCl).

Another aspect of the invention comprises preparing the compound of Formula IA by:

cyclizing a compound of Formula IC in the presence of a diazotizing reagent:

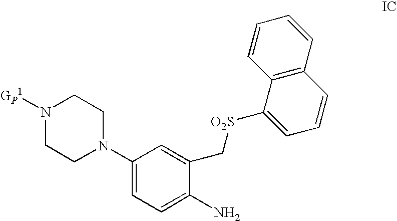

to form a compound of formula ID:

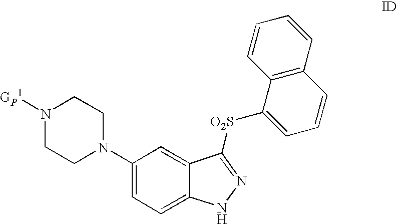

wherein if G_{P}¹ is the same protecting group as G_{P} in the compound of Formula IA, then the compound of Formula IA is formed; or

if G_{P}¹ is a different protecting group from G_{P} in the compound of Formula IA, then the process further comprises:

deprotecting the compound of Formula ID to form a compound of Formula IE:

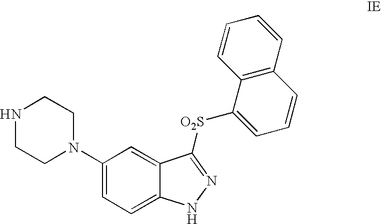

and

protecting compound of Formula IE, thereby forming the compound of Formula IA.

In another embodiment, the diazotizing reagent is sodium nitrate (NaNO₂). In another embodiment, the cyclizing step is performed at less than about 20° C. in the presence of HCl.

In another embodiment, G_{P}¹ is a different protecting group from G_{P} in the compound of Formula IA; and the deprotecting step comprises contacting the compound of Formula ID with an acid. More particularly, the protecting step comprises, reacting activated-G_{P} with the compound of Formula IE. More particular still, activated-G_{P} is selected from the group consisting of halo-G_{P}, tosylate-G_{P}, G_{P}-anhydride, mesylate-G_{P} or triflate-G_{P}.

In another embodiment, G_{P} is Boc and G_{P}¹ is Cbz.

In another embodiment, the process further comprises preparing the compound of Formula IC by:

reducing a compound of Formula IF:

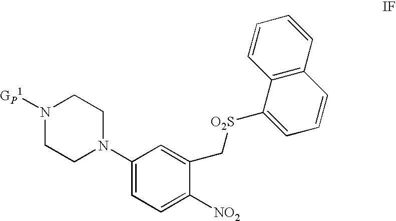

wherein the compound of Formula IC is formed.

In another embodiment, the reducing step is performed in the presence of tin chloride (SnCl₂) and HCl.

In another embodiment, the process further comprises:

reacting 1-(5-fluoro-2-nitrobenzylsulfonyl)naphthalene with a compound of Formula IG:

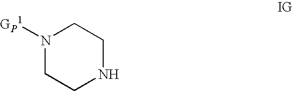

to form the compound of Formula IF.

In another embodiment, the reacting step is performed at greater than about 50° C. in the presence of a base.

Another aspect of the invention further comprises preparing 1-(5-fluoro-2-nitrobenzylsulfonyl)naphthalene by:

reacting 1-fluoro-4-nitrobenzene with 1-(chloromethylsulfonyl)naphthalene.

In another embodiment, the reacting step is performed at less than about 0° C. in the presence of a base.

In another embodiment, any of the foregoing process steps are performed in a protic solvent, an aprotic solvent, a polar solvent, a nonpolar solvent, a protic polar solvent, an aprotic nonpolar solvent, or an aprotic polar solvent.

In another embodiment, any of the foregoing process steps comprises a purification step comprising at least one of: filtration, extraction, chromatography, trituration, or recrystallization.

In another embodiment, any of the foregoing process steps comprises an analytical step comprising liquid chromatography (LC), mass spectroscopy (MS), liquid chromatography/mass spectroscopy (LC/MS), gas chromatography (GC), gas chromatography/mass spectroscopy (GC/MS), nuclear magnetic resonance (NMR), thin layer chromatography (TLC), melting point (MP) analysis, optical rotation (OR) or elemental analysis.

The compounds prepared by these processes are useful in vitro or in vivo in modulating 5HT₆ activity. In a particular embodiment, modulating, refers to inhibiting activity. The compounds may be used alone or in compositions together with a pharmaceutically acceptable carrier or excipient. Pharmaceutical compositions of the present invention comprise a therapeutically effective amount of a compound described herein formulated together with one or more pharmaceutically acceptable carriers.

As used herein, the term “pharmaceutically acceptable carrier” means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. Other suitable pharmaceutically acceptable excipients are described in “Remington's Pharmaceutical Sciences,” Mack Pub. Co., New Jersey, 1991, incorporated herein by reference.

The compounds of the present invention may be administered to humans and other animals orally, parenterally, sublingually, by aerosolization or inhalation spray, rectally, intracisternally, intravaginally, intraperitoneally, bucally, intrathecally or topically in dosage unit formulations containing conventional nontoxic pharmaceutically acceptable carriers, adjuvants, and vehicles as desired. Topical administration may also involve the use of transdermal administration such as transdermal patches or ionophoresis devices. The term parenteral as used herein includes subcutaneous injections, intravenous, intramuscular, intrasternal injection, or infusion techniques.

Methods of formulation are well known in the art and are disclosed, for example, in Remington: The Science and Practice of Pharmacy, Mack Publishing Company, Easton, Pa., 19th Edition (1995). Pharmaceutical compositions for use in the present invention can be in the form of sterile, non-pyrogenic liquid solutions or suspensions, coated capsules, suppositories, lyophilized powders, transdermal patches or other forms known in the art.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-propanediol or 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid find use in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution that, in turn, may depend upon crystal size and crystalline form. Alternatively, dissolving or suspending the drug in an oil vehicle may accomplish delayed absorption of a parenterally administered drug form. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations may also be prepared by entrapping the drug in liposomes or microemulsions, which are compatible with body tissues.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, acetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

The active compounds can also be in microencapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, EtOAc, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulations, ear drops, and the like are also contemplated as being within the scope of this invention.

Compositions of the invention may also be formulated for delivery as a liquid aerosol or inhalable dry powder. Liquid aerosol formulations may be nebulized predominantly into particle sizes that can be delivered to the terminal and respiratory bronchioles.

Effective amounts of the compounds of the invention generally include any amount sufficient to detectably modulate 5HT₆ activity, or by alleviation of symptoms of CNS diseases associated with 5HT₆ activity or susceptible to 5HT₆ activity modulation. The amount of active ingredient that may be combined with the carrier materials to produce a single dosage form will vary depending upon the host treated and the particular mode of administration. It will be understood, however, that the specific dose level for any particular subject in need thereof will depend upon a variety of factors including the activity of the specific compound employed, the age, body weight, general health, sex, diet, time of administration, route of administration, rate of excretion, drug combination, and the severity of the particular disease undergoing therapy. The therapeutically effective amount for a given situation can be readily determined by routine experimentation and is within the skill and judgment of the ordinary clinician.

In another aspect of the invention, kits that include one or more compounds of the invention are provided. Representative kits include a 5HT₆ inhibitor compound of the invention (e.g., a compound of Formula I) and a package insert or other labeling including directions for treating a CNS disease by administering an effective amount of a compound of the present invention.

It is understood that the invention is not limited to the embodiments set forth herein for illustration, but embraces all such forms thereof as come within the scope of the above disclosure.

### EXAMPLES

The following abbreviations are used herein and have the indicated definitions: BOC is t-butoxycarbonyl, Boc₂O is di-tert-butyl dicarbonate or Boc anhydride, and Celite™ is flux-calcined diatomaceous earth. Celite™ is a registered trademark of World Minerals Inc. DMF is N,N-dimethylformamide, EDTA is ethylenediaminetetraacetic acid, EtOAc is ethyl acetate, and EtOH is ethanol. [³H]-LSD is tritium-labeled lysergic acid diethylamide, KOₜBu or KOtBu is potassium tertiary butoxide, PBS is phosphate-buffered saline (pH 7.4), PVT WGA SPA is polyvinyltoluidene wheat germ agglutinin scintillation proximity assay, and THF is tetrahydrofuran.

Referring to the examples that follow, compounds of the present invention were synthesized using the methods described herein, or other methods, which are known in the art.

The compounds and/or intermediates were characterized by LC mass spectrometric analysis performed on an Agilent HPLC and a Hewlett-Packard mess spectrometer with an Onyx Monolithic C18 Column (100×3.0 mm): solvent system: 10-100% Acetonitrile in water, flow rate: 1.8 mL/min, molecular weight range 200-700. Nuclear magnetic resonance (NMR) analysis was performed on the compounds with a 400 MHz Varian NMR instrument (Palo Alto, Calif.). The spectral reference is either TMS or the known chemical shift of the solvent. Some compound samples are run at elevated temperatures (e.g., 75° C.) to promote increased sample solubility.

It should be understood that the organic compounds according to the invention may exhibit the phenomenon of tautomerism. As the chemical structures within this specification can only represent one of the possible tautomeric forms, it should be understood that the invention encompasses any tautomeric form of the drawn structure.

### Example 1

### 1-Isobutyl-3-(naphthalen-1-ylsulfonyl)-5-(piperazin-1-yl)-1H-indazole dihydrochloride

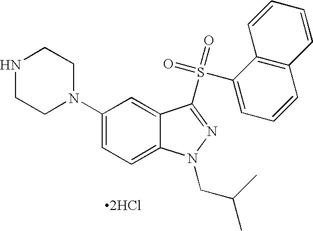

### Step 1: 1-(5-fluoro-2-nitrobenzylsulfonyl)naphthalene

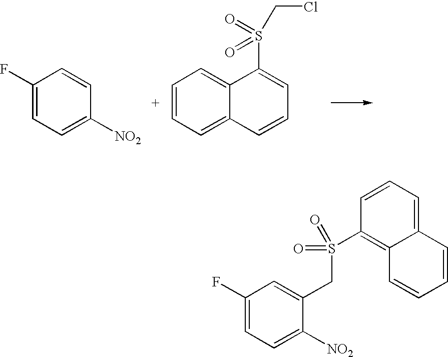

To a stirred mixture of 4-fluoronitrobenzene (Aldrich, 4.80 g, 34.1 mmol) and 1-(chloromethylsulfonyl)naphthalene (Aldrich, 8.20 g, 34.1 mmol) in THF (75 mL) at −78° C. was slowly added KOtBu (1 M in THF, 75 mL). The stirred mixture was allowed to warm to room temperature for 2 hours, quenched with water, and concentrated. The residue was taken into water and extracted with EtOAc. The organic layer was dried over Na₂SO₄, and concentrated in vacuo to provide the title compound (9.50 g, 81%), characterized by NMR and mass spectral analyses. MS (ES⁺) m/e 346 (MH⁺).

### Step 2: benzyl 4-(3-((naphthalen-1-ylsulfonyl)methyl)-4-nitrophenyl)piperazine-1-carboxylate

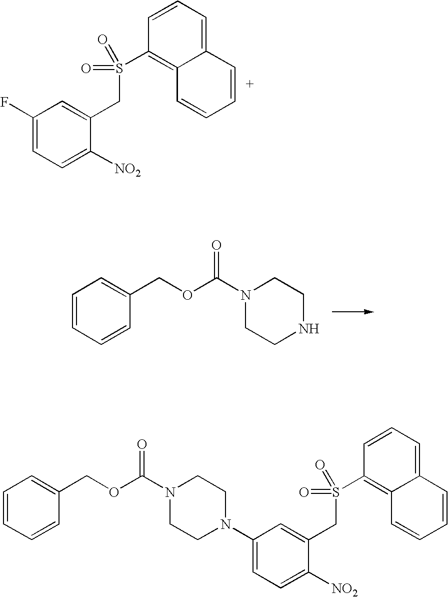

A mixture of benzyl piperazine-1-carboxylate (Aldrich, 3.19 g, 14.5 mmol), 1-(5-fluoro-2-nitrobenzylsulfonyl)naphthalene (5.00 g, 14.5 mmol), and K₂CO₃ (4.00 g, 29.0 mmol) in DMF (32 mL) was stirred at 100° C. for 3 hours, cooled to room temperature, diluted with water, and extracted with EtOAc. The organic layer was washed with water (3×), dried over Na₂SO₄, and concentrated in vacuo to provide the title compound (7.82 g, 94%), characterized by NMR and mass spectral analyses. MS (ES⁺) m/e 546 (MH⁺).

### Step 3: benzyl 4-(4-amino-3-((naphthalen-1-ylsulfonyl)methyl)phenyl)piperazine-1-carboxylate

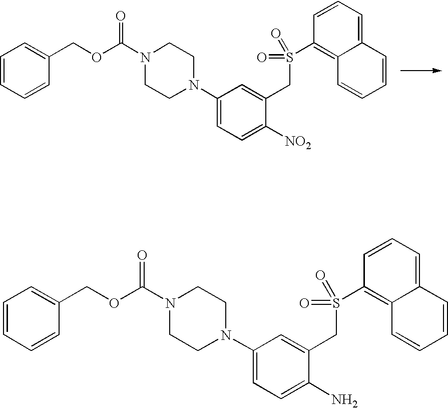

A mixture of benzyl 4-(3-((naphthalen-1-ylsulfonyl)methyl)-4-nitrophenyl)piperazine-1-carboxylate (7.82 g, 14.3 mmol), SnCl₂ (16.2 g, 71.7 mmol), and concentrated HCl (1.8 mL) in EtOH (191 mL) was heated at 80° C. overnight, diluted with CH₂Cl₂, neutralized with Na₂CO₃ to basic condition. The mixture was passed through a pad of Celite™. The solution was concentrated in vacuo to provide the crude title compound (7.72 g), which was carried forward into the next step of the reaction without further purification. MS (ES⁺) m/e 516 (MH⁺).

### Step 4: benzyl 4-(3-(naphthalen-1-ylsulfonyl)-1H-indazol-5-yl)piperazine-1-carboxylate

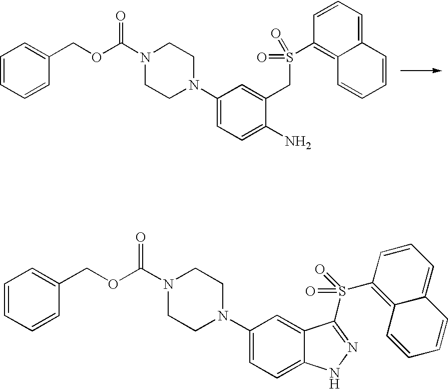

To a solution of crude benzyl 4-(4-amino-3-((naphthalen-1-ylsulfonyl)methyl)phenyl)piperazine-1-carboxylate (7.72 g) in 1 N HCl (249 mL) and MeOH (500 mL) at 0° C. was added NaNO₂ solution (2.07 g, 29.9 mmol in 20 mL water) by drops over a period of 10 minutes. The mixture was stirred for an additional 30 minutes, carefully neutralized with NaHCO₃ solution to pH 10. The brown precipitate was filtered and washed with water and dried in vacuo to provide the title compound (5.8 g, 77% overall for steps 3 and 4), characterized by NMR and mass spectral analyses. MS (ES⁺) m/e 527 (MH⁺).

### Step 5: 3-(naphthalen-1-ylsulfonyl)-5-(piperazin-1-yl)-1H-indazole

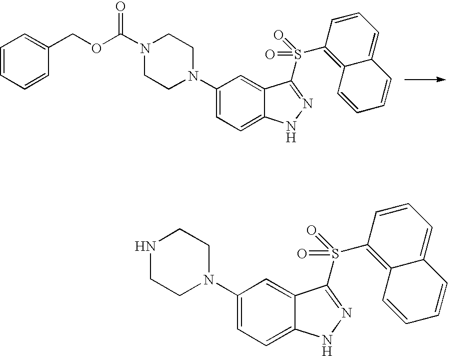

A mixture of benzyl 4-(3-(naphthalen-1-ylsulfonyl)-1 H-indazol-5-yl)piperazine-1-carboxylate (2.50 g, 4.75 mmol) and 30% HBr in HOAc (13.6 mL) was stirred at room temperature for 2 hours. Et₂O (80 mL) was added. The brown precipitate was filtered, washed with Et₂O, treated with NaOH (1 N, 20 mL), extracted with 20% MeOH in EtOAc. Combined extracts were dried over Na₂SO₄ and concentrated in vacuo to provide the title compound (1.48 g, 80%), characterized by NMR and mass spectral analyses. MS (ES⁺) m/e 593 (MH⁺).

### Step 6: tert-butyl 4-(3-(naphthalen-1-ylsulfonyl)-1H-indazol-5-yl)piperazine-1-carboxylate

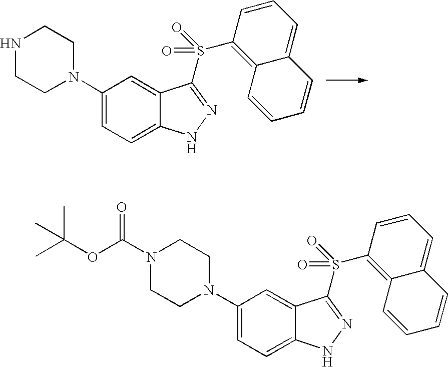

A mixture of 3-(naphthalen-1-ylsulfonyl)-5-(piperazin-1-yl)-1H-indazole dihydrochloride salt (2.00 g, 4.00 mmol), Boc₂O (1.02 g, 4.66 mmol), and triethylamine (1.65 g, 16.3 mmol) in DMF (15.5 mL) was heated at 50° C. overnight, diluted with water, extracted with EtOAc. The organic layer was dried over Na₂SO₄ and concentrated in vacuo to provide the title compound (2.00 g, 99%), characterized by NMR and mass spectral analyses. MS (ES⁺) m/e 493 (MH⁺).

### Step 7: tert-butyl 4-(1-isobutyl-3-(naphthalen-1-ylsulfonyl)-1H-indazol-5-yl)piperazine-1-carboxylate

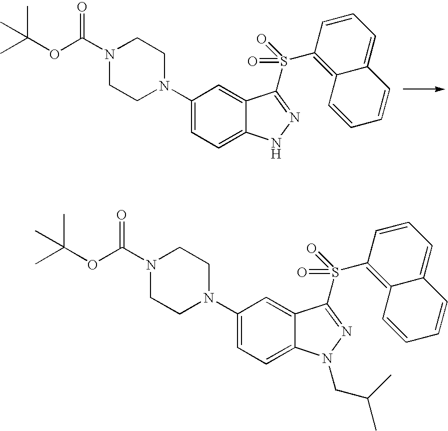

To a solution of tert-butyl 4-(3-(naphthalen-1-ylsulfonyl)-1H-indazol-5-yl)piperazine-1-carboxylate (0.50 g, 1.02 mmol) in DMF (5.1 mL) was added KOtBu (1 M THF solution, 1.5 mL) followed by isobutyl iodide (0.139 g, 1.02 mmol). The mixture was stirred overnight, diluted with water and extracted with EtOAc. The organic layer was washed with water (3×), dried over Na₂SO₄, concentrated in vacuo, and purified by chromatography with EtOAc/Hexanes to provide the title compound (0.401 g, 73%), characterized by NMR and mass spectral analyses. MS (ES⁺) m/e 549 (MH⁺).

### Step 8: 1-isobutyl-3-(naphthalen-1-ylsulfonyl)-5-(piperazin-1-yl)-1H-indazole dihydrochloride

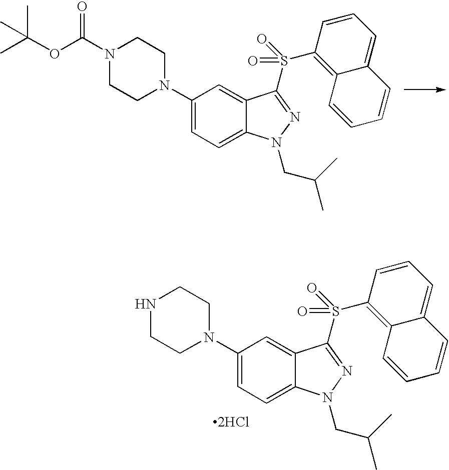

tert-Butyl 4-(1-isobutyl-3-(naphthalen-1-ylsulfonyl)-1 H-indazol-5-yl)piperazine-1-carboxylate (0.40 g, 0.73 mmol) was treated with TFA (20 mL) at room temperature for 1 hour. The reaction mixture was concentrated to dryness. The residue was dissolved in EtOAc and was added Et₂O. The precipitate was filtered and washed with Et₂O and dried in vacuo to provide the title compound (0.34 g, 69%), characterized by NMR and mass spectral analyses. ¹H NMR (400 MHz, CDCl₃) δ ppm 0.66 (d, J=6.6 Hz, 6H) 2.05-2.12 (m, 1H) 3.20 (b, 4H) 3.31-3.34 (m, 4H) 4.23 (d, J=7.2 Hz, 2H) 7.12 (d, J=2.0 Hz, 1H) 7.33 (dd, J=9.4 and 2.2 Hz, 1H) 7.54 (m, 2H), 7.70-7.74 (m, 2H) 8.00-8.02 (m, 1H) 8.23 (d, J=8.4 Hz, 1H) 8.54 (dd, J=7.4 and 1.0 Hz, 1H) 8,75 (dd, J=9.6 and 1.6 Hz, 1H) 9.29 (b, 2H). MS (ES⁺) m/e 449 (MH⁺).

### Example 2

### 1-Benzyl-3-(naphthalene-1-sulfonyl)-5-piperazin-1-yl-1H-indazole dihydrochloride

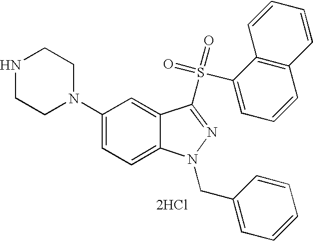

The title compound was prepared using essentially the same procedure as described in Example 1 and the benzyl chloride as the alkylating agent of step 7 (0.35 g, 66% yield) ¹H NMR (400 MHz, CDCl₃) δ ppm 3.29 (b, 4H) 3.23-3.36 (m, 4H) 7.10-7.20 (m, 5H) 7.32 (dd, J=9.4 and 2.3 Hz, 1H) 7.58-7.64 (m, 2H) 7.69-7.74 (m, 2H) 8.00-8.04 (m, 1H) 8.25 (d, J=8.2 Hz, 1H) 8.55 (dd, J=7.4 and 1.0 Hz, 1H) 8.75-8.78 (m, 1H), 9.23 (b, 2H). MS (ES⁺) m/e 483 (MH⁺).

### Example 3

### 1-(3-Chlorobenzyl)-5-(piperazin-1-yl)-3-(naphthalen-1-ylsulfonyl)-1H-indazole dihydrochloride

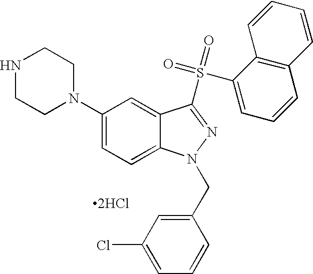

### Step 1: 1-methyl-4-(4-nitrophenyl)piperazine

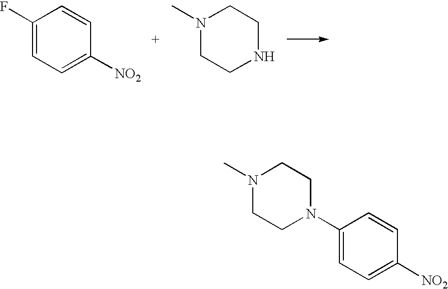

A mixture of 1-methylpiperazine (3.55 g, 35.5 mmol), 4-fluoronitrobenzene (5.00 g, 35.5 mmol), and K₂CO₃ (9.80 g, 71.0 mmol) in DMF (71 mL) was stirred at 100° C. for 3 hours, cooled to room temperature, diluted with water, and extracted with EtOAc. The organic layer was washed with water (3×), dried over Na₂SO₄, and concentrated in vacuo to provide the title compound (7.84 g, 89%), characterized by NMR and mass spectral analyses. MS (ES⁺) m/e 222 (MH⁺).

### Step 2: 1-methyl-4-(3-((naphthalen-1-ylsulfonyl)methyl)-4-nitrophenyl)piperazine

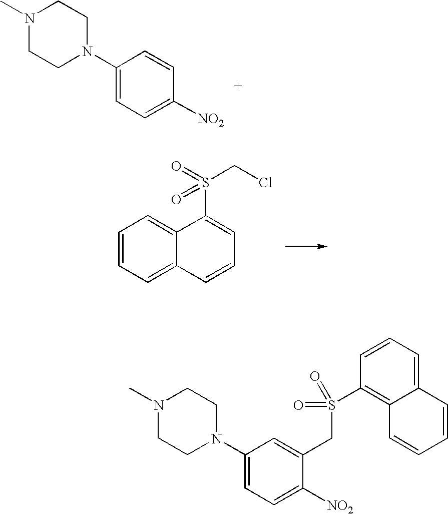

To a stirred mixture of 1-methyl-4-(4-nitrophenyl)piperazine (4.00 g, 18.1 mmol) and 1-(chloromethylsulfonyl)naphthalene (4.35 g, 18.1 mmol) in THF (40 mL) at −78° C. was slowly added KOtBu (1 M in THF, 40 mL). The stirred mixture was allowed to warm to room temperature for 2 hours, quenched with water, and concentrated. The residue was taken into water and extracted with EtOAc. The organic layer was dried over Na₂SO₄, and concentrated in vacuo to provide the title compound (6.70 g, 87%), characterized by NMR and mass spectral analyses. MS (ES⁺) m/e 426 (MH⁺).

### Step 3: 4-(4-methylpiperazin-1-yl)-2-((naphthalen-1-ylsulfonyl)methyl)aniline

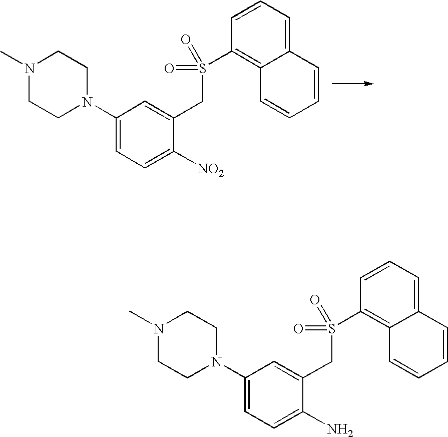

A mixture of 1-methyl-4-(3-((naphthalen-1-ylsulfonyl)methyl)-4-nitrophenyl)piperazine (6.70 g, 15.7 mmol) and tin foil (10.7 g, 90.4 mmol) in concentrated HCl (107 mL) and MeOH (107 mL) was heated at reflux for 30 minutes, cooled to room temperature, carefully neutralized with NaOH solution until pH=10. The mixture was diluted with CH₂Cl₂, passed through a pad of Celite™, concentrated in vacuo to provide the title compound (5.00 g, 70%), characterized by NMR and mass spectral analyses. MS (ES⁺) m/e 396 (MH⁺).

### Step 4: 5-(4-methylpiperazin-1-yl)-3-(naphthalen-1-ylsulfonyl)-1H-indazole

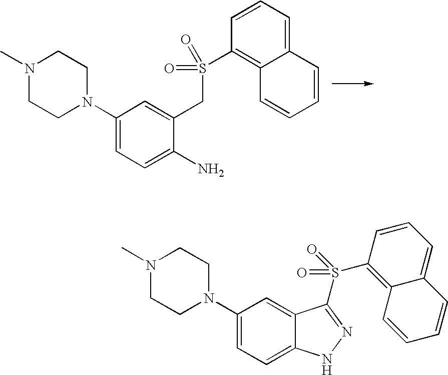

To a solution of 4-(4-methylpiperazin-1-yl)-2-((naphthalen-1-ylsulfonyl)methyl)aniline (5.00 g, 12.6 mmol) in 1 N HCl (210 mL) at 0° C. was added NaNO₂ solution (1.31 g, 19.0 mmol in 15 mL water) by drops over a period of 10 minutes. The mixture was stirred for an additional 30 minutes, carefully neutralized with NaHCO₃ solution to pH˜10. The brown precipitate was filtered and washed with water and dried in vacuo to provide the title compound (4.70 g, 91%), characterized by NMR and mass spectral analyses. MS (ES⁺) m/e 407 (MH⁺).

### Step 5: 1-(3-chlorobenzyl)-5-(4-methylpiperazin-1-yl)-3-(naphthalen-1-ylsulfonyl)-1H-indazole

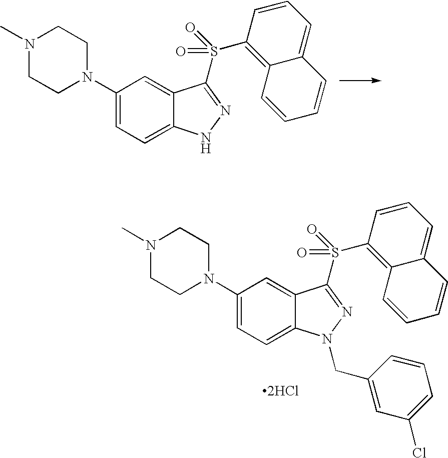

To a solution of 5-(4-methylpiperazin-1-yl)-3-(naphthalen-1-ylsulfonyl)-1H-indazole (0.20 g, 0.45 mmol) in DMF (1.5 mL) was added NaH (0.043 g, 1.81 mmol). The mixture was stirred for 10 minutes followed by addition of 3-chlorobenzyl chloride (0.093 g, 0.45 mmol). The reaction mixture was stirred overnight, diluted with water, and extracted with EtOAc. The organic layer was washed with water (3×), dried over Na₂SO₄, and concentrated in vacuo, and purified by chromatography with MeOH/CH₂Cl₂ to provide the title compound (75 mg, 32%), characterized by NMR and mass spectral analyses. MS (ES⁺) m/e 532 (MH⁺).

### Step 6: 1-(3-chlorobenzyl)-5-(piperazin-1-yl)-3-(naphthalen-1-ylsulfonyl)-1H-indazole dihydrochloride

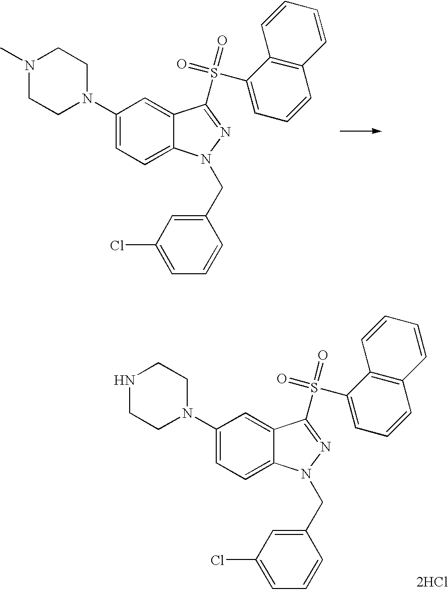

A mixture of 1-(3-chlorobenzyl)-5-(4-methylpiperazin-1 -yl)-3-(naphthalen-1-ylsulfonyl)-1H-indazole (37 mg, 0.07 mmol), 1-chloroethyl chloroformate (100 mg, 0.70 mmol), and 1,8-diaminonaphthalene (75 mg, 0.35 mmol) was heated at 110° C. in a pressure tube for 2 hours, cooled to room temperature, treated with 10% water in dioxane, concentrated, and purified by reverse phase HPLC to provide the title compound (25 mg, 69%), characterized by NMR and mass spectral analyses. ¹H NMR (400 MHz, CDCl₃) δ ppm 3.21-3.35 (m, 8H) 5.70 (s, 2H) 7.06 (d, J=7.4 Hz, 1H) 7.17-7.26 (m, 2H) 7.35 (dd, J=9.4 and 2.2 Hz, 1H) 7.57-7.61 (m, 2H) 7.69-7.78 (m, 2H) 8.01-8.04 (m, 1H) 8.26 (d, J=8.4 Hz, 1H) 8.5 (d, J=6.3 Hz, 1H) 8.74-8.76 (m, 1H) 8.92 (b, 2H). MS (ES⁺) m/e 517 (MH⁺).

### Example 4

### 1-(3-(Naphthalen-1-ylsulfonyl)-5-(piperazin-1-yl)-1H-indazol-1-yl)ethanone hydrochloride

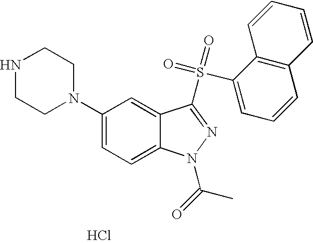

The title compound was prepared using essentially the same procedure and acetyl chloride as an acylation agent as described in Example 1. (33%) ¹H NMR (400 MHz, CDCl₃) δ ppm 2.64 (s, 3H) 3.24-3.37 (m, 8H) 7.24 (d, J=2.2 Hz, 1H) 7.49 (dd, J=9.3 and 2.4 Hz, 1H) 7.64 (dd, J=8.2 and 1.1 Hz, 1H) 7.70-7.78 (m, 2H) 8.08 (d, J=8.0 Hz, 1H) 8.14 (d, J=9.3 Hz, 1H) 8.35 (d, J=8.3 Hz, 1H) 8.62 (d, J=7.5 Hz, 1H) 8.64 (b, 2H) 8.80 (d, J=8.7 Hz, 1H). MS (ES⁺) m/e 435 (MH⁺).

### Example 5

### 1-Methyl-3-(naphthalen-1-ylsulfonyl)-5-(piperazin-1-yl)-1H-indazole dihydrochloride

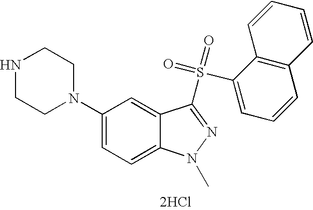

The title compound was prepared using essentially the same procedure as described in Example 1 and methyl iodide as an alkylating agent. (61%) ¹H NMR (400 MHz, CDCl₃) δ ppm 3.32-3.52 (m, 8H) 4.03 (s, 3H) 7.22 (d, J=2.0 Hz, 1H) 7.37 (dd, J=9.4 and 2.3 Hz, 1H) 7.55-7.73 (m, 2H) 8.02 (d, J=8.7 Hz, 1H) 8.26 (d, J=8.2 Hz, 1H) 8.51 (d, J=7.4 Hz, 1H) 8.75 (d, J=8.5 Hz, 1H) 9.18 (b, 2H). MS (ES⁺) m/e407 (MH⁺).

### Example 6

### 1-Ethyl-3-(naphthalen-1-ylsulfonyl)-5-(piperazin-1-yl)-1H-indazole dihydrochloride

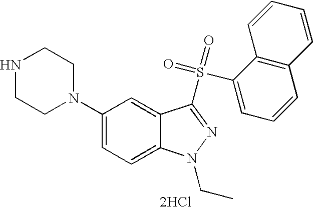

The title compound was prepared using essentially the same procedure as described in Example 1 and ethyl iodide as an alkylating agent. (57%). ¹H NMR (400 MHz, CDCl₃) δ ppm 1.29 (t, J=7.2 Hz, 3H) 3.2 (b, 4H) 3.30-3.32 (m, 4H) 4.43 (t, J=7.2 Hz, 2H) 7.16 (d, J=2.0 Hz, 1H) 7.34 (dd, J=9.3 and 2.1 Hz, 1H) 7.55-7.73 (m, 4H) 8.02 (d, J=7.6 Hz, 1H) 8.25 (d, J=8.2 Hz, 1H) 8.52 (dd, J=7.4 and 1.2 Hz, 1H) 8.79 (d, J=8.6 Hz, 1H) 9.10 (b, 2H). MS (ES⁺) m/e 421 (MH⁺).

### Example 7

### 3-(Naphthalen-1-ylsulfonyl)-5-(piperazin-1-yl)-1-propyl-1H-indazole dihydrochloride

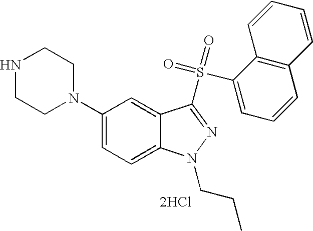

The title compound was prepared using essentially the same procedure as described in Example 1 and propyl iodide as an alkylating agent. (70%). ¹H NMR (400 MHz, CDCl₃) δ ppm 0.62 (t, J=7.6 Hz, 3H) 1.73 (q, J=7.0 Hz, 2H) 3.21 (b, 4H) 3.26-3.32 (m, 4H) 4.38 (t, J=6.7 Hz, 2H) 7.14 (d, J=2.1 Hz, 1H) 7.33 (dd, J=9.4 and 2.2 Hz, 1H) 7.55-7.73 (m, 4H) 8.02 (d, J=9.3 Hz, 1H) 8.25 (d, J=8.4 Hz, 1H) 8.53 (dd, J=7.3 and 1.2 Hz, 1H) 8.76 (d, J=8.6 Hz, 1H) 9.09 (b, 2H). MS (ES⁺) m/e 435 (MH⁺).

### Example 8

### 1-Isopropyl-3-(naphthalen-1-ylsulfonyl)-5-(piperazin-1-yl)-1H-indazole dihydrochloride

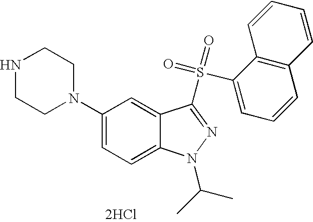

The title compound was prepared using essentially the same procedure as described in Example 1 and isopropyl iodide as an alkylating agent. (30%). ¹H NMR (400 MHz, CDCl₃) δ ppm 1.40 (d, J=6.6 Hz, 6H) 3.21-3.30 (m, 8H) 4.99-5.03 (m, 1H) 7.11 (d, J=2.0 Hz, 1H) 7.31 (dd, J=9.3 and 2.2 Hz, 1H) 7.55-7.74 (m, 4H) 8.01 (d, J=8.0 Hz, 1H) 8.24 (d, J=8.2 Hz, 1H) 8.52 (dd, J=7.4 and 1.1 Hz, 1H) 8.86 (d, J=8.8 Hz, 1H) 8.98 (b, 2H). MS (ES⁺) m/e 435 (MH⁺).

### Compound C: 3-(naphthalen-1-ylsulfonyl)-5-(piperazin-1-yl)-1H-indazole dihydrochloride

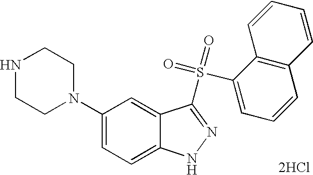

Compound C is prepared as described in the Examples (particularly Example 53) in U.S. Pat. No. 7,238,696, the entire contents of which is hereby incorporated by reference as if set forth fully herein.

### Example 9

### 5HT₆ Binding Affinity

CHO-Dukx-A2 cells expressing the serotonin 5HT6 receptor subtype (clone 50-7) grown adherently in 10-cell stacks are detached and harvested in PBS buffer containing 5 mM EDTA using conventional cell harvesting protocols, followed by centrifugation at 2000 rpms for 10 minutes (supernatant discarded) or provided as wet cell pellets by Applied Cell Sciences (Rockville, Md.). The pellets are gently resuspended with enough PBS buffer containing MgCl₂ and CaCl₂, (GIBCO 14040-133) to achieve a final concentration of ˜40×10⁶ cells/ml. The cell suspension is aliquoted into microfuge tubes, centrifuged at 2000 rpms for 10 minutes, supernatant discarded and stored as dry pellets at −80° C.

Protein is measured in 5 μl lysate mixed with 200 μl diluted Bradford reagent, using Bovine Plasma Gamma globulin as a standard.

Binding experiments are performed in a total volume of 200 μl using a 96 well microtiter plate format (Packard Optiplate). On the day of the assay, the cells are thawed and resuspended with enough assay buffer (i.e. PBS containing MgCl₂ and CaCl₂, (GIBCO 14040-133) supplemented with additional MgCl₂ to achieve a final concentration of 10 mM) to achieve 40-80 μg or 100-200 K cells/well. To each well of the microtiter plate, 20 μl of 10× test compound in water containing 3.3% DMSO, 3 nM of [3H]-LSD (GE, SA: 80 Ci/mmol), cells and assay buffer are combined to achieve a volume of 150 μl. Assay buffer and 10 μM cold methiothepin are substituted for the test compound in separate wells to define ‘total’ and ‘nonspecific’ binding, respectively. The incubation period is initiated by the addition of 50 μl of 10 mg/ml PVT WGA SPA beads (RPNQ0060, Amersham GE Healthcare) mixed in assay buffer for a final concentration of 1 mg/well. The plates are sealed and gently shaken at room temperature using an orbital shaker (setting 1.5) until equilibrium is achieved (2-6 hours). Radioactivity (CPM) is measured by Packard TopCount (1 minutes counting time/well).

Specific binding is described as the total radioactivity bound less the amount bound in the presence of 10 μM methiothepin, referred to as nonspecific binding (NSB). Binding in the presence of varying concentrations of test compounds is expressed as percent of specific binding in the absence of compound:

{(Bound−NSB)/(Total−NSB)}×100=% Total

Regression analysis of % bound data from ten concentrations is performed in GraphPad Prism, XL Fit or equivalent software. IC₅₀ values are calculated using a four-parameter logistic curve fitting model and Ki values are calculated by the Cheng-Prusoff equation below:

Ki=IC₅₀/(1+L/Kd)

where L is the nM concentration of the radioactive ligand used and the Kd is the dissociation constant of the ligand for the receptor. The Kd for [³H]-LSD in the SPA binding format is ˜3 nM. Using this assay, the following Ki values listed in Table 1 were determined. Brain/plasma and bioavailability values were obtained according to Example 10.

TABLE 1

| | | Brain/ | |
|---|---|---|---|
| Example | 5HT₆ Ki | plasma | Oral bio- |
| No. | (nM) | ratio | availability |
| 1 | 4.6 | 0.81 | 22% |
| 2 | 7.5 | 0.26 | 50% |
| 3 | 23.5 | | |
| 4 | 1.0 | | |
| 5 | 1.6 | 0.3 | |
| 6 | 1.9 | 0.45 | |
| 7 | 2.2 | 0.54 | |
| 8 | 3 | 0.36 | |
| C | 0.8 | <0.01 | 3% |

### Example 10

### Pharmacokinetics and CNS Penetration of Compounds were Determined Using HPLC Analysis of the Prepared Samples and Presented in Tables 2, 3, & 4 Below

Male Long Evans Rats were dosed with the candidate drug substance at the dosage amount and route listed above using standard pharmacokinetic procedures (Applied Biopharmaceutics & Pharmacokinetics, Shargel, L and Yu, A, 4^{th} Ed 1999, McGraw-Hill, N.Y.; Welling, P G, Pharmacokinetics, Process and Mathematics, ACS Monograph 185, American Chemical Society 1986, Washington, D.C.; Lee, P and Amidon, G L, Pharmacokinetic Analysis-A Practical Approach, Technomic Pub Co Lancaster Pa. 1996)).

TABLE 2
Pharmacokinetics in Male Long Evans Rats (n = 3)
following a single IV bolus at 2 mg/kg

| | AUC_{0-inf} |
|---|---|
| compounds | (hr * ng/mL) |
| Compound C | 390 |
| Example 1 | 378 |
| Example 2 | 843 |

TABLE 3
Pharmacokinetics in Male Long Evans Rats following
a single oral dose of 10 mg/kg

| | AUC_{0-inf} | Bioavailability |
|---|---|---|
| compounds | (hr * ng/mL) | (%) |
| Compound C | 119 | 3 |
| Example 1 | 413 | 22 |
| Example 2 | 2095 | 50 |

TABLE 4
CNS penetration in Male Long Evans Rats (n = 3) following
a single intraperitoneal dose of 10 mg/kg

| compound | matrix | AUCₗₐₛₜ (ng/mL) | Brain/plasma ratio |
|---|---|---|---|
| COMPOUND C | Brain | Not detectable | <0.01 |
| | plasma | 119 | |
| Example 1 | brain | 397 | 0.81 |
| | plasma | 491 | |
| Example 2 | brain | 159 | 0.26 |
| | plasma | 623 | |

PK Results: As evidenced by the pharmacokinetic (PK) data provided in Tables 1, 2, 3 and 4, the screened compounds of the present invention have superior bioavailability and blood brain barrier penetration profiles than compared to Compound C.

### Example 11

### Microsomal Stability

Compounds listed in Table 5, column A, are prepared as described in U.S. Pat. No. 7,238,696. Each of the compounds in Table 5, columns A and B, were screen according to the microsomal stability procedure described in Di et al., Journal of Biomolecular Screening 2003:453-462.

TABLE 5
Microsomal stability comparison of compounds
having 1-substituted-piperazinyl groups (A)
and compounds with no substitution
on the piperazinyl group (B).

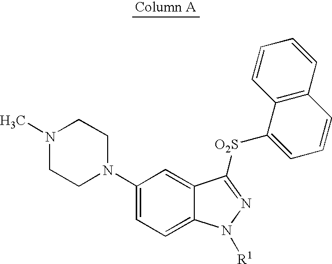

Microsomal Stability
(min)

| R¹ | Rat | Human |
|---|---|---|
| isobutyl | 2 | 2 |
| benzyl | 2 | 2 |
| 3-chlorobenzyl | * | * |
| acetyl | * | * |
| methyl | * | * |
| ethyl | * | * |
| n-propyl | * | * |
| isopropyl | 3 | * |

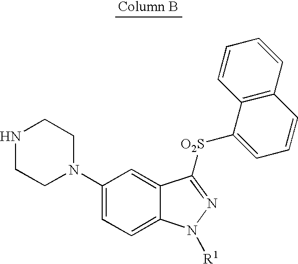

Microsomal Stability
(min)

| R¹ | Rat | Human |
|---|---|---|
| isobutyl | 24 | >30 |
| (Example 1) | | |
| benzyl | 25 | 13 |
| (Example 2) | | |
| 3-chlorobenzyl | * | * |
| (Example 3) | | |
| acetyl | * | * |
| (Example 4) | | |
| methyl | * | * |
| (Example 5) | | |
| ethyl | * | * |
| (Example 6) | | |
| n-propyl | * | * |
| (Example 7) | | |
| isopropyl | >30 | >30 |
| (Example 8) | | |

(* = No data)

Stability Results: As evidenced by the microsomal stability data provided in Table 5, the unsubstituted-piperazinyl compounds listed in column B have greater microsomal stability than their 1-substituted-piperazinyl homologues in column A.

Each of the above-cited references are hereby incorporated by reference as if set forth fully herein.

What is claimed:

1. A compound of Formula I: 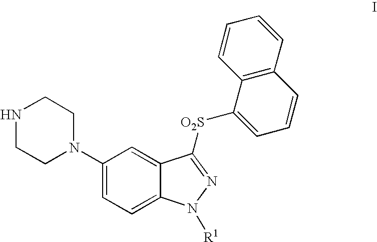
wherein,
R¹ is selected from the group consisting of C₆-C₁₀ aryl, C₁-C₆ alkyl, C₇-C₁₄ arylalkyl and C₁-C₆ acyl, each substituted with 0-4 substituents independently selected from the group consisting of C₁-C₄ alkyl, C₃-C₆ cycloalkyl, C₂-C₆ alkenyl, C₂-C₆ alkynyl, halo, nitro, cyano, hydroxy, phenyl 5-7 membered heterocyclyl, 5-7 membered heteroaryl, —N(R^{a})₂, —C(O)R^{b}, —OR^{c} and —S(O)ₚR^{d};
each R^{a} is independently H, C₁-C₄ alkyl optionally substituted with halo, —CHO, —C(O)(C₁-C₄ alkyl), or —CO₂(C₁-C₄ alkyl);
each R^{b} is independently H, —OH, —O(C₁-C₄), C₁-C₄ alkyl optionally substituted with halo, —NH₂, —NH(C₁-C₄ alkyl), or —N(C₁-C₄ alkyl)₂;
each R^{c} is independently H, C₁-C₄ alkyl optionally substituted with halo, —CHO or —C(O)(C₁-C₄ alkyl);
each R^{d} is independently C₁-C₄ alkyl optionally substituted with halo or —OH; and
each p is independently 0, 1 or 2; or
a tautomer or pharmaceutically acceptable salt thereof.

2. The compound of claim 1, wherein R¹ is C₁-C₆ alkyl, C₁-C₆ acyl, or C₇-C₉ arylalkyl, each optionally substituted with 1, 2, or 3 halo; or a tautomer or pharmaceutically acceptable salt thereof.

3. The compound of claim 1, wherein R¹ is C₁-C₆ alkyl or C₇-C₉ arylalkyl, each optionally substituted with 1, 2, or 3 halo; or a tautomer or pharmaceutically acceptable salt thereof.

4. The compound of claim 1, wherein R¹ is C₁-C₆ alkyl; or a tautomer or pharmaceutically acceptable salt thereof.

5. The compound of claim 4, wherein R¹ is methyl, ethyl, n-propyl, isopropyl, n-butyl, isobutyl, or tert-butyl; or a tautomer or pharmaceutically acceptable salt thereof.

6. The compound of claim 5, wherein R¹ is methyl, ethyl, n-propyl, or isopropyl; or a tautomer or pharmaceutically acceptable salt thereof.

7. The compound of claim 3, wherein R¹ is C₇-C₉ arylalkyl optionally substituted with 1, 2, or 3 halo; a tautomer or pharmaceutically acceptable salt thereof.

8. The compound of claim 7, wherein R¹ is benzyl optionally substituted with 1, 2, or 3 halo; or a tautomer or pharmaceutically acceptable salt thereof.

9. The compound of claim 1, selected from the group consisting of: 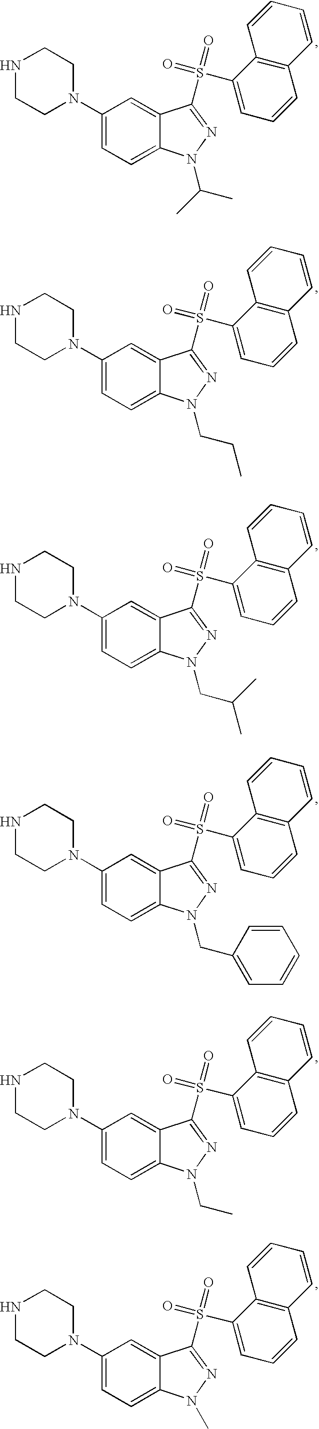 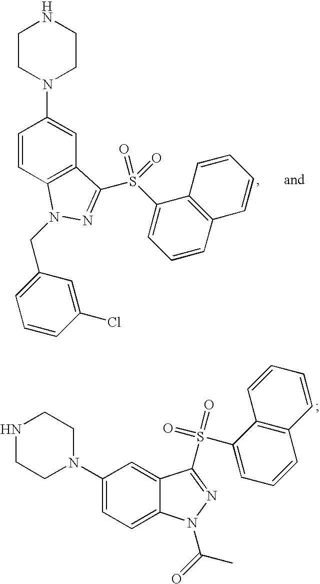
or a tautomer or pharmaceutically acceptable salt thereof.

10. The compound of claim 1, having the structure: 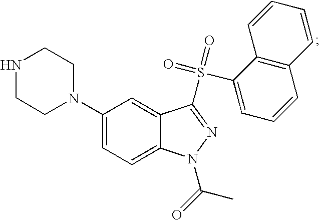
or a tautomer or pharmaceutically acceptable salt thereof.

11. The compound of claim 1, having the structure: 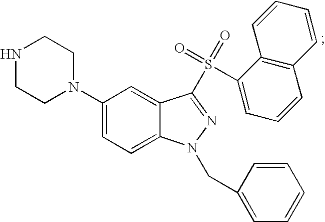
or a tautomer or pharmaceutically acceptable salt thereof.

12. The compound of claim 1, having the structure: 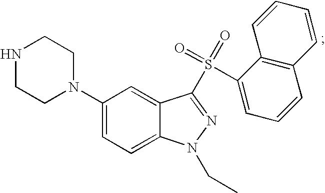
or a tautomer or pharmaceutically acceptable salt thereof.

13. The compound of claim 1, having the structure: 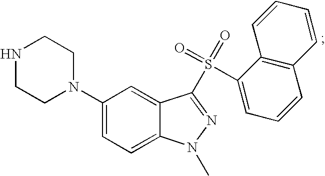
or a tautomer or pharmaceutically acceptable salt thereof.

14. The compound of claim 1, having the structure: 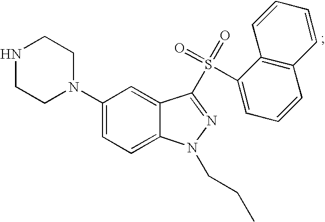
or a tautomer or pharmaceutically acceptable salt thereof.

15. The compound of claim 1, having the structure: 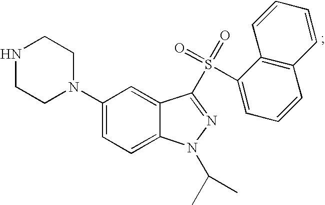
or a tautomer or pharmaceutically acceptable salt thereof.

16. The compound of claim 1, wherein the pharmaceutically acceptable salt is the hydrochloride (HCl).

17. A composition comprising a compound of claim 1 and a pharmaceutically acceptable carrier.

18. A method of modulating 5HT₆ receptor activity in a subject, the method comprising administering to the subject in need thereof in need thereof a compound of Formula I: 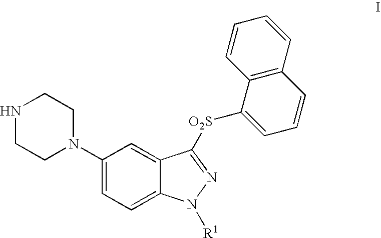
wherein,
R¹ is selected from the group consisting of C₆-C₁₀ aryl, C₁-C₆ alkyl, C₇-C₁₄ arylalkyl and C₁-C₆ acyl, each substituted with 0-4 substituents independently selected from the group consisting of C₁-C₄ alkyl, C₃-C₆ cycloalkyl, C₂-C₆ alkenyl, C₂-C₆ alkynyl, halo, nitro, cyano, hydroxy, phenyl, 5-7 membered heterocyclyl, 5-7 membered heteroaryl, —N(R^{a})₂, —C(O)R^{b}, —OR^{c} and —S(O)ₚR^{d};
each R^{a} is independently H, C₁-C₄ alkyl optionally substituted with halo, —CHO, —C(O)(C₁-C₄ alkyl), or —CO₂(C₁-C₄ alkyl);
each R^{b} is independently H, —OH, —O(C₁-C₄), C₁-C₄ alkyl optionally substituted with halo, —NH₂, —NH(C₁-C₄ alkyl), or —N(C₁-C₄ alkyl)₂;
each R^{c} is independently H, C₁-C₄ alkyl optionally substituted with halo, —CHO or —C(O)(C₁-C₄ alkyl);
each R^{d} is independently C₁-C₄ alkyl optionally substituted with halo or —OH; and
each p is independently 0, 1 or 2; or
a tautomer or pharmaceutically acceptable salt thereof;
wherein 5HT₆ receptor activity is modulated in the subject.

19. The method of claim 18, wherein the compound is administered parenterally to the subject.

20. The method of claim 18, wherein the compound is capable of crossing the blood brain barrier in the subject.

21. The method of claim 18, wherein the compound is administered in an amount sufficient to provide about 1 to about 4000 ng/mL of the compound to brain: tissue, plasma, blood, central spinal fluid (CSF), or intraspinal fluid (ISF) of the subject within 24 hours after administration.

22. The method of claim 18, wherein the subject is suffering from psychoses, anxiety, depression, epilepsy obsessive compulsive disorders, migraine, cognitive disorders, sleep disorders, feeding disorders, anorexia, bulimia, binge eating disorders, panic attacks, disorders resulting from withdrawal from drug abuse, schizophrenia, gastrointestinal disorders, irritable bowel syndrome, memory disorders, Alzheimer's disease, Parkinson's disease, Huntington's chorea, schizophrenia, attention deficit hyperactive disorder, neurodegenerative diseases characterized by impaired neuronal growth, or pain.

23. A method of treating a central nervous system (CNS) disease or disorder comprising administering to the subject in need thereof a compound of Formula I: 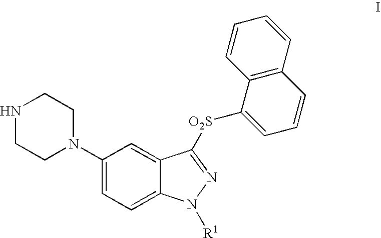
wherein,
R¹ is selected from the group consisting of C₆-C₁₀ aryl, C₁-C₆ alkyl, C₇-C₁₄ arylalkyl and C₁-C₆ acyl, each substituted with 0-4 substituents independently selected from the group consisting of C₁-C₄ alkyl, C₃-C₆ cycloalkyl, C₂-C₆ alkenyl, C₂-C₆ alkynyl, halo, nitro, cyano, hydroxy, phenyl, 5-7 membered heterocyclyl, 5-7 membered heteroaryl, —N(R^{a})₂, —C(O)R^{b}, —OR^{c} and —S(O)ₚR^{d};
each R^{a} is independently H, C₁-C₄ alkyl optionally substituted with halo, —CHO, —C(O)(C₁-C₄ alkyl), or —CO₂(C₁-C₄ alkyl);
each R^{b} is independently H, —OH, —O(C₁-C₄), C₁-C₄ alkyl optionally substituted with halo, —NH₂, —NH(C₁-C₄ alkyl), or —N(C₁-C₄ alkyl)₂;
each R^{c} is independently H, C₁-C₄ alkyl optionally substituted with halo, —CHO or —C(O)(C₁-C₄ alkyl);
each R^{d} is independently C₁-C₄ alkyl optionally substituted with halo or —OH; and
each p is independently 0, 1 or 2; or
a tautomer or pharmaceutically acceptable salt thereof.

24. The method of claim 23, wherein the disease or disorder is psychoses, anxiety, depression, epilepsy obsessive compulsive disorders, migraine, cognitive disorders, sleep disorders, feeding disorders, anorexia, bulimia, binge eating disorders, panic attacks, disorders resulting from withdrawal from drug abuse, schizophrenia, gastrointestinal disorders, irritable bowel syndrome, memory disorders, Alzheimer's disease, Parkinson's disease, Huntington's chorea, schizophrenia, attention deficit hyperactive disorder, neurodegenerative diseases characterized by impaired neuronal growth, or pain.

25. A process for the preparation of a compound of Formula I: 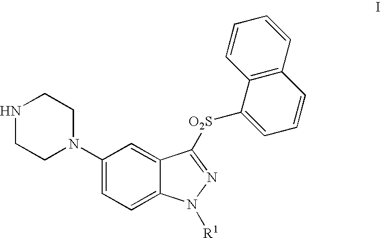
wherein,
R¹ is selected from the group consisting of C₆-C₁₀ aryl, C₁-C₆ alkyl, C₇-C₁₄ arylalkyl and C₁-C₆ acyl, each substituted with 0-4 substituents independently selected from the group consisting of C₁-C₄ alkyl, C₃-C₆ cycloalkyl, C₂-C₆ alkenyl, C₂-C₆ alkynyl, halo, nitro, cyano, hydroxy, phenyl, 5-7 membered heterocyclyl, 5-7 membered heteroaryl, —N(R^{a})₂, —C(O)R^{b}, —OR^{c} and —S(O)ₚR^{d}; each R^{a} is independently H, C₁-C₄ alkyl optionally substituted with halo, —CHO, —C(O)(C₁-C₄ alkyl), or —CO₂(C₁-C₄ alkyl);
each R^{b} is independently H, —OH, —O(C₁-C₄), C₁-C₄ alkyl optionally substituted with halo, —NH₂, —NH(C₁-C₄ alkyl), or —N(C₁-C₄ alkyl)₂;
each R^{c} is independently H, C₁-C₄ alkyl optionally substituted with halo, —CHO or —C(O)(C₁-C₄ alkyl);
each R^{d} is independently C₁-C₄ alkyl optionally substituted with halo or —OH; and
each p is independently 0, 1 or 2; or
a tautomer or pharmaceutically acceptable salt thereof
the process comprising:
(a) reacting R¹-G_{A} or R¹═O with a compound of Formula IA: 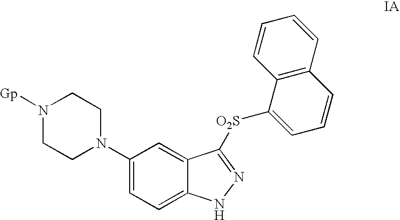
wherein,
G_{A} is an activating group; and
G_{P} is a protecting group;
to form a compound of Formula IB: 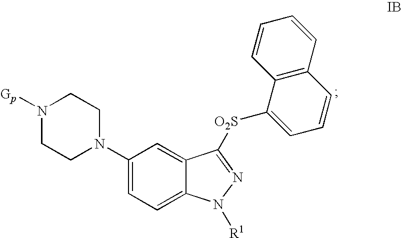
and
(b) deprotecting the compound of Formula IB.

26. The process of claim 25, wherein G_{A} is halo, tosylate, mesylate or triflate.

27. The process of claim 26, wherein G_{A} is iodo.

28. The process of claim 25, wherein G_{P} is t-butoxycarbonyl (Boc), benzyl, acetyl, p-methoxybenzyl (PMB), C₁-C₆ alkyl 9-fluoroenylmethoxycarbonyl (Fmoc), benzyloxycarbonyl (Cbz), trifluoroacetyl, tosyl or trityl.

29. The process of claim 25, wherein the reacting step is performed in the presence of a base.

30. The process of claim 29, wherein the base is potassium tertiary butoxide (KOₜBu).

31. The process of claim 25, wherein the deprotecting step is performed in the presence of an acid.

32. The process of claim 31, wherein the acid is trifluoroacetic acid (TFA) or hydrochloric acid (HCl).

33. The process of claim 32, further comprising preparing the compound of Formula IA by:
(a) cyclizing a compound of Formula IC in the presence of a diazotizing reagent: 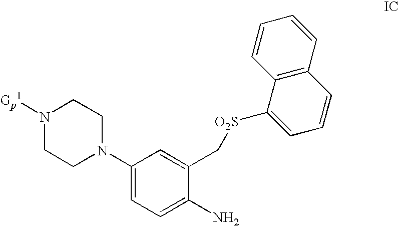
to form a compound of formula ID: 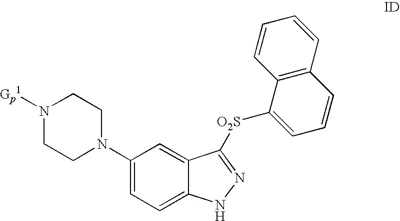
wherein if G_{P}¹ is the same protecting group as G_{P} in the compound of Formula IA, then the compound of Formula IA is formed; or
if G_{P}¹ is a different protecting group from G_{P} in the compound of Formula IA, then the process further comprises:
(b) deprotecting the compound of Formula ID to form a compound of Formula IE: 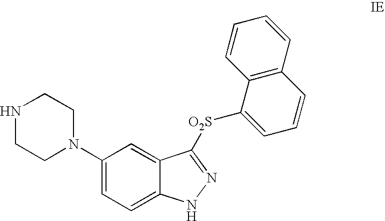
and protecting compound of Formula IE, thereby forming the compound of Formula IA.

34. The process of claim 33, wherein the diazotizing reagent is sodium nitrate (NaNO₂).

35. The process of claim 34, wherein the cyclizing step is performed at less than about 20° C. in the presence of HCl.

36. The process of claim 33, wherein G_{P}¹ is a different protecting group from G_{P} in the compound of Formula IA; and the deprotecting step comprises contacting the compound of Formula ID with an acid.

37. The process of claim 36, wherein the protecting step comprises, reacting activated-G_{P} with the compound of Formula IE.

38. The process of claim 37, wherein activated-G_{P} is selected from the group consisting of halo-G_{P}, tosylate-G_{P}, G_{P}-anhydride, mesylate-G_{P} or triflate-G_{P}.

39. The process of claim 33, wherein G_{P} is Boc and G_{P}¹ is Cbz.

40. The process of claim 33, wherein the process further comprises preparing the compound of Formula IC by: reducing a compound of Formula IF: 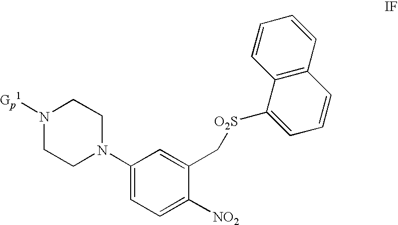
wherein the compound of Formula IC is formed.

41. The process of claim 40, wherein the reducing step is performed in the presence of tin chloride (SnCl₂) and HCl.

42. The process of claim 40, wherein the process further comprises:
reacting 1-(5-fluoro-2-nitrobenzylsulfonyl)naphthalene with a compound of Formula IG: 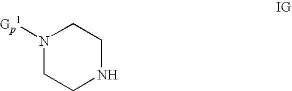
to form the compound of Formula IF.

43. The process of claim 42, wherein the reacting step is performed at greater than about 50° C. in the presence of a base.

44. The process of claim 42, further comprising preparing 1-(5-fluoro-2-nitrobenzylsulfonyl)naphthalene by: reacting 1-fluoro-4-nitrobenzene with 1-(chloromethylsulfonyl)naphthalene.

45. The process of claim 44, wherein the reacting step is performed at less than about 0° C. in the presence of a base.

46. The process of claim 25, wherein any of the process steps are performed in a protic solvent, an aprotic solvent, a polar solvent, a nonpolar solvent, a protic polar solvent, an aprotic nonpolar solvent, or an aprotic polar solvent.

47. The process of claim 25, wherein any of the process steps comprises a purification step comprising at least one of: filtration, extraction, chromatography, trituration, or recrystallization.

48. The process of claim 25, wherein any of the process steps comprises an analytical step comprising liquid chromatography (LC), mass spectroscopy (MS), liquid chromatography/mass spectroscopy (LC/MS), gas chromatography (GC), gas chromatography/mass spectroscopy (GC/MS), nuclear magnetic resonance (NMR), thin layer chromatography (TLC), melting point (MP) analysis, optical rotation (OR) or elemental analysis.
